(19) United States
(12) Patent Application Publication
(10) Pub. No.: US 2015/0051210 A1
(43) Pub. Date: Feb. 19, 2015

(54) Tyrosine Kinase Inhibitor Combinations and their Use

(71) Applicants: Ralph Tiedt, Prattein (CH); Alan Buckler, Arlington, MA (US); Fred Harbinski, Cambridge, MA (US); Sneha Sanghavi, Cambridge, MA (US); Douglas Jeffery, Cambridge, MA (US); Christopher Wilson, Reading, MA (US)

(72) Inventors: Ralph Tiedt, Prattein (CH); Alan Buckler, Arlington, MA (US); Fred Harbinski, Cambridge, MA (US); Sneha Sanghavi, Cambridge, MA (US); Douglas Jeffery, Cambridge, MA (US); Christopher Wilson, Reading, MA (US)

(73) Assignee: Novartis AG, Basel (CH)

(21) Appl. No.: 14/388,251

(22) PCT Filed: Apr. 1, 2013
(86) PCT No.: PCT/US13/34759

§ 371 Date: Sep. 26, 2014

Related U.S. Application Data
(60) Provisional application No. 61/619,502, filed on Apr. 3, 2012.

(51) Int. Cl. A61K 31/53; A61K 31/506; A61K 31/5025

(52) U.S. Cl. CPC A61K 31/53; A61K 31/5025; A61K 31/506
USPC 514/243; 514/248

(57) ABSTRACT

The invention relates to pharmaceutical combination product comprising (i) a MET inhibitor and (ii) an FGFR inhibitor, or a pharmaceutical acceptable salt thereof, respectively, or a prodrug thereof, respectively, and at least one pharmaceutically acceptable carrier.

### SUMMARY OF THE INVENTION

The present invention relates to pharmaceutical combinations comprising of (i) a MET inhibitor and (ii) an FGFR inhibitor, or a pharmaceutically acceptable salt thereof, respectively, or a prodrug thereof, which are jointly active in the treatment of proliferative diseases, corresponding pharmaceutical formulations, uses, methods, processes, commercial packages and related invention embodiments.

### BACKGROUND OF THE INVENTION

The proto-oncogen cMET (MET) encodes the protein Hepatocyte Growth Factor Receptor (HGFR) which has tyrosine kinase activity and is essential for embryonic development and wound healing. Upon Hepatocyte Growth Factor (HGF) stimulation, MET induces several biological responses, leading to invasive growth. Abnormal MET activation triggers tumor growth, formation of new blood vessels (angiogenesis) and metastasis, in various types of malignancies, including cancers of the kidney, liver, stomach, breast and brain. A number of MET kinase inhibitors are known, and alternatively inhibitors of HGF-induced MET (=HGFR) activation. The biological functions of c-MET (or c-MET signaling pathway) in normal tissues and human malignancies such as cancer have been well documented (Christensen, J. G. et al., Cancer Lett. 2005, 225(1):1-26; Corso, S. et al., Trends in Mol. Med. 2005, 11(6):284-292).

So far, several distinct membrane FGFRs with tyrosine kinase activity have been identified in vertebrates and all of them belong to the tyrosine kinase superfamily: FGFR1 (=CD331, see also Fibroblast growth factor receptor 1); FGFR2 (=CD332, see also Fibroblast growth factor receptor 2); FGFR3 (=CD333, see also Fibroblast growth factor receptor 3); FGFR4 (=CD334 see also Fibroblast growth factor receptor 4); and FGFR6.

Epidemiological studies have reported genetic alterations and/or abnormal expression of FGFs/FGFRs in human cancers: translocation and fusion of FGFR1 to other genes resulting in constitutive activation of FGFR1 kinase is responsible for 8p11 myeloproliferative disorder (MacDonald D & Cross NC, Pathobiology 74:81-8 (2007)). Gene amplification and protein over-expression have been reported for FGFR1, FGFR2 and FGFR4 in breast tumors (Adnane J et al., Onco-gene 6:659-63 (1991); Jaakkola S etal., Int. J. Cancer54:378-82 (1993); Penault-Llorca F etal., Int. J. Cancer 61: 170-6 (1995); Reis-Filho J S et al., Clin. Cancer Res. 12:6652-62 (2006)). So-matic activating mutations of FGFR2 are known in gastric (Jang J H etal., Cancer Res. 61:3541-3 (2001)) and endometrial cancers (Pollock P M etal., Oncogene (May 21, 2007)). Recurrent chromosomal translocations of 4p16 into the immunoglobuling heavy chain switch region at 14q32 result in deregulated over-expression of FGFR3 in multiple myeloma (Chesi M etal, Nature Genetics 16:260-264 (1997); Chesi M etal., Blood 97:729-736 (2001)) and somatic mutations in specific domains of FGFR3 leading to ligand-independent constitutive activation of the receptor have been identified in urinary bladder carcinomas and multiple myelomas (Cappellen D etal., Nature Genetics 23:18-20 (1999); Billerey C etal., Am. J. Pathol. 158(6):1955-9 (2001); van Rhijn B W G et al., Eur. J. Hum. Genet. 10: 819-824 (2002); Ronchetti C et al., Oncogene 20: 3553-3562 (2001)).

### GENERAL DESCRIPTION OF THE INVENTION

Using cancer cells originally dependent on either MET or FGFR, surprisingly a by-pass of dependence through ligand-mediated activation of alternative receptor tyrosine kinases (RTKs) was observed. By-pass mechanisms were discovered when treating MET- or FGFR-dependent lines with a corresponding selective inhibitor (that is, MET-dependent lines with a MET inhibitor and FGFR-dependent lines with an FGFR inhibitor) and at the same time adding supernatants from cells transfected with cDNA coding for various secreted proteins It could be shown that the MET and FGFR RTKs can compensate for the loss of function of the other due to inhibition, thus leading to “rescue” of proliferating cells if only one of these RTKs is inhibited by an appropriate drug. This allows to deduce a general concept and teaching that a combination of FGFR and MET inhibitors wiil enable the effective treatment of diseases where activity of MET compensates for inhibition of FGFR and/or activity of FGFR compensates for MET inhibition.

It was thus found that combined inhibition of these RTKs can lead to synergistic anti-cancer activity especially when MET and an FGFR RTK are both active and then, according to the invention, can be inhibited simultaneously or jointly sequentially.

### SPECIFIC DESCRIPTION OF THE INVENTION

The present invention, according to a first embodiment, relates to a pharmaceutical combination, comprising (i) a MET inhibitor and (ii) an FGFR inhibitor, or a pharmaceutically acceptable salt thereof, respectively, or a prodrug thereof, respectively, and at least one pharmaceutically acceptable carrier.

A further embodiment of this invention provides a combination, comprising, a quantity which is jointly therapeutically effective against an FGFR tyrosine kinase activity and/or MET tyrosine kinase activity mediated disease, especially a cancer, (i) FGFR tyrosine kinase inhibitor and (ii) MET tyrosine kinase inhibitor, or, respectively, a pharmaceutically acceptable salt thereof, and at least one pharmaceutically acceptable carrier.

A further embodiment relates to the use of the inventive combination for treating an FGFR tyrosine kinase activity and/or MET tyrosine kinase activity mediated disease, especially a cancer.

A further embodiment relates to the use of a combination of (i) an FGFR tyrosine kinase inhibitor and (ii) a MET tyrosine kinase inhibitor or, respectively, a pharmaceutically acceptable salt thereof, for the manufacture of a medicament or a pharmaceutical product for treating an FGFR tyrosine kinase activity and/or MET tyrosine kinase activity mediated disease, especially a cancer.

A further embodiment relates to a method of treating an FGFR tyrosine kinase activity and/or MET tyrosine kinase activity mediated disease, especially a cancer, with a combination of (i) an FGFR tyrosine kinase inhibitor and (ii) a MET tyrosine kinase inhibitor or, respectively, a pharmaceutically acceptable salt thereof.

A further embodiment relates to a method for the treatment of an FGFR tyrosine kinase activity and/or MET tyrosine kinase activity mediated disease, especially a cancer, said method comprising administering an effective amount of a combi-nation of or a combination product comprising (i) an FGFR tyrosine kinase inhibitor and (ii) a MET tyrosine kinase inhibitor to a subject in need thereof, such as a warm-blooded animal, in particular a human.

Yet a further embodiment of present invention relates to a pharmaceutical product or a commercial package comprising a combination according to the invention described herein, in particular together with instructions for simultaneous, separate or sequential use (especially for being jointly active) thereof in the treatment of an FGFR tyrosine kinase activity and/or MET tyrosine kinase activity mediated disease, especially a cancer, in particular for use in the treatment of an FGFR tyrosine kinase activity and/or MET tyrosine kinase activity mediated disease, especially a cancer.

A further embodiment of present invention relates to the use of (i) an FGFR tyrosine kinase inhibitor and (ii) a MET tyrosine kinase inhibitor or, respectively, a pharmaceutically acceptable salt thereof, for the preparation of a combination product according to present invention.

WO 2011/018454 discloses MET tyrosine kinase inhibitors. Especially useful is the compound with the name (E)-2-(1-(3-((7-fluoroquinolin-6-yl)methyl)imidazo[1,2-b]pyridazin-6-yl)ethylidene)hydrazinecarboxamide (also called Cpd. A in the following) having the formula:

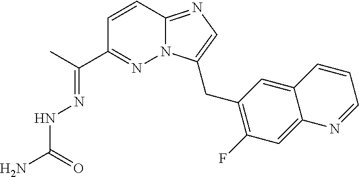 [SMILES: C/C(=N\NC(N)=O)c1ccc2ncc(Cc3cc4cccnc4cc3F)n2n1]

### see WO 11 018454, Example 1.

WO 2008/064157 discloses MET tyrosine kinase inhibitors, especially useful is the compound with the name 2-fluoro-N-methyl-4-[(7-quinolin-6-yl-methyl)-imidazo[1,2-b]triazin-2-yl]benzamide (also named Cpd. B hereinafter) which has the formula

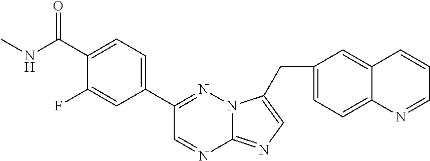 [SMILES: CNC(=O)c1ccc(-c2cnc3ncc(Cc4ccc5ncccc5c4)n3n2)cc1F]

### see WO 2008/064157, Example 7.

Other MET inhibitors, their pharmaceutically acceptable salts, and prodrugs thereof, (which also includes compounds or antibodies active against HGF) are exemplified as below:

Crizotinib (Pfizer) (aka PF02341066) having the formula

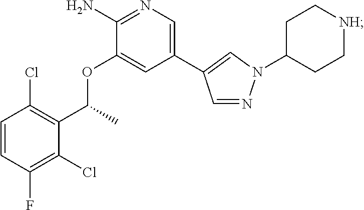 [SMILES: C[C@@H](Oc1cc(-c2cnn(C3CCNCC3)c2)cnc1N)c1c(Cl)ccc(F)c1Cl]

cabozantinib (Exelixis) (aka XL-184) having the formula

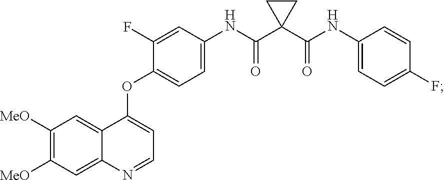 [SMILES: COc1cc2nccc(Oc3ccc(NC(=O)C4(C(=O)Nc5ccc(F)cc5)CC4)cc3F)c2cc1OC]

tivatinib (ArQule, daiichi, Kyowa) (aka ARQ-197) having the formula

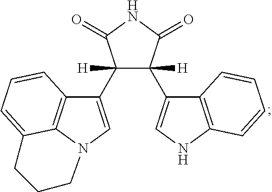 [SMILES: O=C1NC(=O)[C@@H](c2cn3c4c(cccc24)CCC3)[C@H]1c1c[nH]c2ccccc12]

foretinib (Exelixis, GlaxoSmithKline) (aka XL-880) having the formula

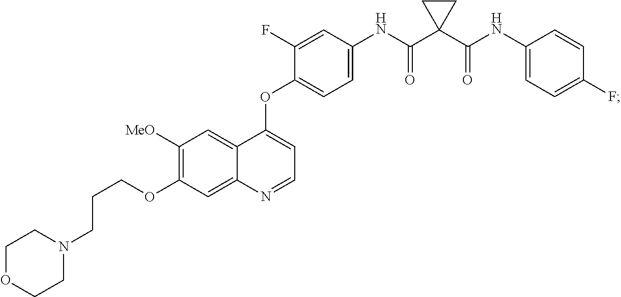 [SMILES: COc1cc2c(Oc3ccc(NC(=O)C4(C(=O)Nc5ccc(F)cc5)CC4)cc3F)ccnc2cc1OCCCN1CCOCC1]

MGCD-265 (MethylGene) having the formula

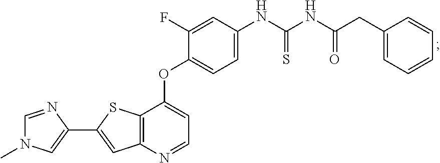 [SMILES: Cn1cnc(-c2cc3nccc(Oc4ccc(NC(=S)NC(=O)Cc5ccccc5)cc4F)c3s2)c1]

AMG-208 (Amgen) (see also WO 2008/008539) having the formula

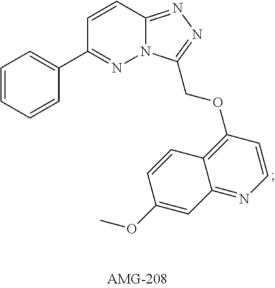 [SMILES: COc1ccc2c(OCc3nnc4ccc(-c5ccccc5)nn34)ccnc2c1]

AMG-337 (Amgen);

JNJ-38877605 (Johnson & Johnson) (aka BVT051,see also WO 2007/075567) having the formula

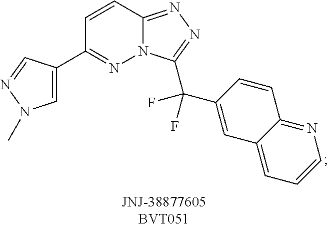 [SMILES: Cn1cc(-c2ccc3nnc(C(F)(F)c4ccc5ncccc5c4)n3n2)cn1]

MK-8033 (Merck & Co) having the formula

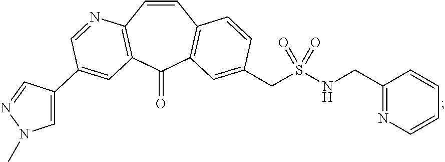 [SMILES: Cn1cc(-c2cnc3ccc4ccc(CS(=O)(=O)NCc5ccccn5)cc4c(=O)c3c2)cn1]

E-7050 (Eisai) having the formula

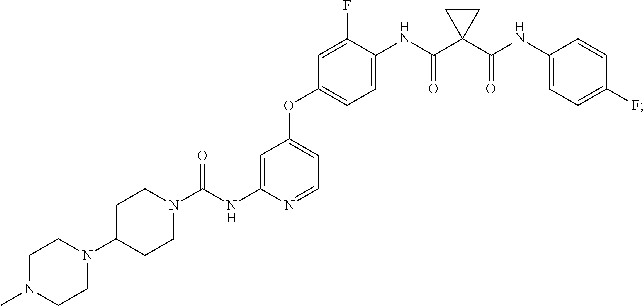 [SMILES: CN1CCN(C2CCN(C(=O)Nc3cc(Oc4ccc(NC(=O)C5(C(=O)Nc6ccc(F)cc6)CC5)c(F)c4)ccn3)CC2)CC1]

EMD-1204831 (Merck Serono);

EMD-1214063 (Merck Serono, see also WO 2007/019933) having the formula

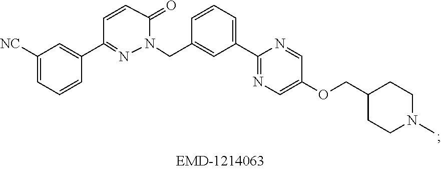 [SMILES: CN1CCC(COc2cnc(-c3cccc(Cn4nc(-c5cccc(C#N)c5)ccc4=O)c3)nc2)CC1]

amuvatinib (SuperGen, aka MP-470) having the formula

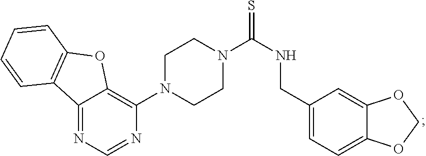 [SMILES: S=C(NCc1ccc2c(c1)OCO2)N1CCN(c2ncnc3c2oc2ccccc23)CC1]

LY-2875358 (Eli Lilly);

BMS-817378 (BristolMyersSquibb, Simcere) having the formula

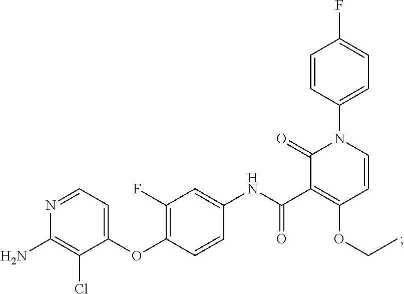 [SMILES: CCOc1ccn(-c2ccc(F)cc2)c(=O)c1C(=O)Nc1ccc(Oc2ccnc(N)c2Cl)c(F)c1]

DP-3590 (Deciphera);

ASP-08001 (Suzhou Ascepion Pharmaceuticals);

HM-5016504 (Hutchison Medipharma);

PF-4217903 (Pfizer, see also U.S. 2007/0265272) having the formula

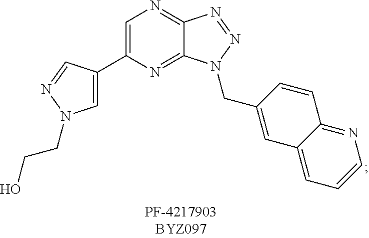 [SMILES: OCCn1cc(-c2cnc3nnn(Cc4ccc5ncccc5c4)c3n2)cn1]

or

SGX523 (SGX,see also WO 2008/051808) having the formula

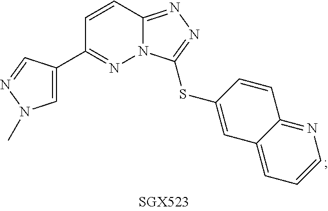 [SMILES: Cn1cc(-c2ccc3nnc(Sc4ccc5ncccc5c4)n3n2)cn1]

or antibodies or related molecules, e.g.

ficlatuzumab (AVEO) monoclonal antibody against HGF; onartuzumab (Roche) monoclonal antibody against MET ; rilotuzumab (Amgen) monoclonal antibody against HGF; Tak-701 (Takeda) monoclonal antibody against HGF); LA-480 (Eli Lilly) monoclonal antibody against MET; and/or LY.2875358 (Eli Lilly) monoclonal antibody against MET.

WO 2006/000420 discloses FGFR tyrosine kinase inhibitors, especially the compounds of formula (II) and salts, esters, N-oxides or prodrugs thereof, are a particular embodiment.

Especially preferred

Is

3-(2,6-dichloro-3,5-dimethoxy-phenyl)-1-{6-[4-(4-ethylpiperazin-1-yl)-phenylamino]-pyrimidin-4-yl}-1-methyl-urea (BGJ398, also named Cpd. C) having the formula:

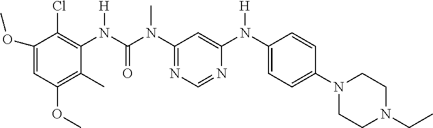 [SMILES: CCN1CCN(c2ccc(Nc3cc(N(C)C(=O)Nc4c(C)c(OC)cc(OC)c4Cl)ncn3)cc2)CC1]

see WO2006/000420, Example 145.

Other FGFR tyrosine kinase inhibitors, or a pharmaceutically accetpable salt or a prodrug thereof include but are not limited to :

AZD-4547 (AstraZeneca) having the formula:

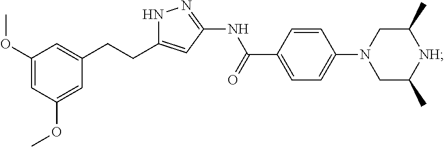 [SMILES: COc1cc(CCc2cc(NC(=O)c3ccc(N4C[C@@H](C)N[C@@H](C)C4)cc3)n[nH]2)cc(OC)c1]

PD173074 (Imperial College London) (N-[2-[[4-(diethylamino)butyl]amino-6-(3,5-dimethoxyphenyl)pyrido[2,3-d]pyrimidin-7-yl]-N′-(1,1-dimethylethyl)urea having the formula:

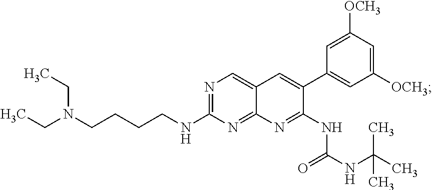 [SMILES: CCN(CC)CCCCNc1ncc2cc(-c3cc(OC)cc(OC)c3)c(NC(=O)NC(C)(C)C)nc2n1]

or less specific FGFR tyrosine kinase inhibitors, e.g. intedanib, dovitinib, brivanib (especially the alaninate), cediranib, masitinib, orantinib, ponatinib and E-7080 of the following formulae:

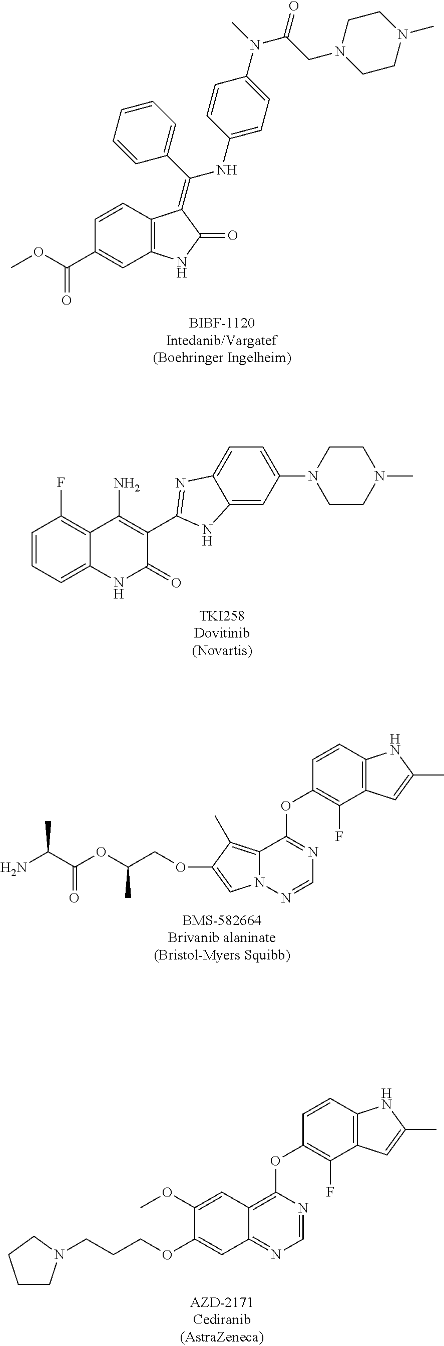

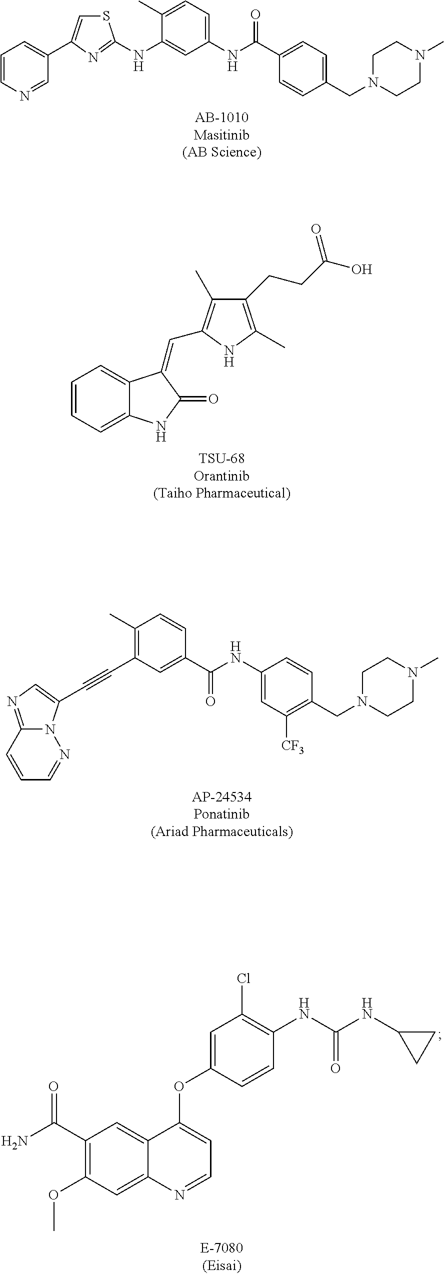 [SMILES: Cc1ccc(NC(=O)c2ccc(CN3CCN(C)CC3)cc2)cc1Nc1nc(-c2cccnc2)cs1 ; Cc1[nH]c(/C=C2\C(=O)Nc3ccccc32)c(C)c1CCC(=O)O ; Cc1ccc(C(=O)Nc2ccc(CN3CCN(C)CC3)c(C(F)(F)F)c2)cc1C#Cc1cnc2cccnn12 ; COc1cc2nccc(Oc3ccc(NC(=O)NC4CC4)c(Cl)c3)c2cc1C(N)=O]

or antibodies or related molecules, e.g. selected from the group consisting of:

HGS1036/FP-1039 (Human Genome Science/Five Prime) (see also J. Clin. Oncol. 28:15s, 2010): soluble fusion protein consisting of the extracellular domains of human FGFR1 linked to the Fc region of human Immunoglobulin G1 (IgG1), designed to seuqester and bind multiple FGF ligands and lock activation of multiple FGF receptors; MFGR1877S (Genentech/Roche): monoclonal antibody; AV-370 (AVEO): humanized antibody; GP369/AV-396b (AVEO): FGFR-IIIb-specific antibody; and HuGAL-FR21 (Galaxy Biotech): monoclonal antibody (FGFR2).

Compounds useful according to the invention can also include all isotopes of atoms occurring in the intermediates or final compounds. Isotopes include those atoms having the same atomic number but different mass numbers. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F ³¹P, ³²P, ³⁵S, ³⁶Cl, ¹²⁵I respectively.

The present invention embodiments also include pharmaceutically acceptable salts of the compounds useful according to the invention described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present invention also includes prodrugs of the compounds useful according to the invention. As used herein, “prodrugs” refer to any covalently bonded carriers which release the active parent drug when administered to a mammalian subject. Prodrugs can be prepared by modifying functional groups present in the compounds in such a way that the modifications are cleaved, either in routine manipulation or in vivo, to the parent compounds. Prodrugs include compounds wherein hydroxyl, amino, sulfhydryl, or carboxyl groups are bonded to any group that, when administered to a mammalian subject, cleaves to form a free hydroxyl, amino, sulfhydryl, or carboxyl group respectively. Examples of prodrugs include, but are not limited to, acetate, formate and benzoate derivatives of alcohol and amine functional groups in the compounds of the invention. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference in their entirety.

The compounds useful according to the invention, as well as their pharmaceutically acceptable salts or prodrugs, can also be present as tautomers, N-oxides or solvates, e.g. hydrates. All these variants, as well as any single one thereof or combination of two or more to less than all such variants, are encompassed and to be read herein where a compound included in the inventive combination products, e.g. an FGFR tyrosine kinase inhibitor and/or a MET tyrosine kinase inhibitor, is mentioned.

The present invention, according to a first embodiment mentioned above and below, relates to a pharmaceutical combination, especially a pharmaceutical combination product, comprising the mentioned combination partners and at least one pharmaceutically acceptable carrier.

“Combination” refers to formulations of the separate partners with or without instructions for combined use or to combination products. The combination partners may thus be entirely separate pharmaceutical dosage forms or pharmaceutical compositions that are also sold independently of each other and where just instructions for their combined use are provided in the package equipment, e.g. leaflet or the like, or in other information e.g. provided to physicians and medical staff (e.g. oral communications, communications in writing or the like), for simultaneous or sequential use for being jointly active, especially as defined below.

“Combination product” refers especially to either a fixed combination in one dosage unit form, or a kit of parts for the combined administration where an FGFR tyrosine kinase inhibitor and a MET tyrosine kinase inhibitor (and optionally yet a further combination partner (e.g. an other drug as explained below, also referred to as “co-agent”) may be administered independently at the same time or separately within time intervals, especially where these time intervals allow that the combination partners show a cooperative (=joint), e.g. synergistic effect. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration and/or at the same time.

The term “combination product” as used herein thus means a pharmaceutical product that results from the mixing or combining of more than one active ingredient and includes both fixed and non-fixed combinations of the active ingredients (which may also be combined).

The term “fixed combination” means that the active ingredients, e.g. an FGFR tyrosine kinase inhibitor and MET tyrosine kinase inhibitor, are both administered to a patient simultaneously in the form of a single entity or dosage. In other terms: the active ingredients are present in one dosage form, e.g. in one tablet or in one capsule.

The term “non-fixed combination” means that the active ingredients are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients. The term “non-fixed combination” thus defines especially a “kit of parts” in the sense that the combination partners (i) FGFR tyrosine kinase inhibitor and (ii) MET tyrosine kinase inhibitor (and if present further one or more co-agents) as defined herein can be dosed independently of each other or by use of different fixed combinations with distinguished amounts of the combination partners, i.e. simultaneously or at different time points, where the combination partners may also be used as entirely separate pharmaceutical dosage forms or pharmaceutical formulations that are also sold independently of each other and just instructions of the possibility of their combined use is or are provided in the package equipment, e.g. leaflet or the like, or in other information e.g. provided to physicians and medical staff. The independent formulations or the parts of the kit of parts can then, e.g. be administered simultaneously or chronologically staggered, that is at different time points and with equal or different time intervals for any part of the kit of parts. Very preferably, the time intervals are chosen such that the effect on the treated disease in the combined use of the parts is larger than the effect which would be obtained by use of only any one of the combination partners (i) and (ii), thus being jointly active. The ratio of the total amounts of the combination partner (i) to the combination partner (ii) to be administered in the combined preparation can be varied, e.g. in order to cope with the needs of a patient sub-population to be treated or the needs of the single patient which different needs can be due to age, sex, body weight, etc. of the patients.

The invention also relates to (i) a MET inhibitor and (ii) an FGFR inhibitor, or a pharmaceutically acceptable salt thereof, for combined use in a method of treating an FGFR tyrosine kinase activity and/or MET tyrosine kinase activity mediated disease, especially a cancer.

In a further embodiment, the MET inhibitor and the FGFR inhibitor for use according to the preceding paragraph are selected as follows: the MET tyrosine kinase inhibitor is selected from the group consisting of (E)-2-(1-(3-((7-fluoroquinolin-6-yl)methyl)imidazo[1,2-b]pyridazin-6-yl)ethylidene)hydrazinecarboxamide and 2-fluoro-N-methyl-4-[(7-quinolin-6-yl-methyl)-imidazo[1,2-b]triazin-2-yl]benzamide, or a pharmaceutically acceptable salt or prodrug thereof, respectively, and the FGR-R tyrosine kinase inhibitor is 3-(2,6-dichloro-3,5-dimethoxy-phenyl)-1-{6-[4-(4-ethyl-piperazin-1-yl)-phenylamino]-pyrimidin-4-yl}-1-methyl-urea, or a pharmaceutically acceptable salt or prodrug thereof.

The combination partners (i) and (ii) in any invention embodiment are preferably formulated or used to be jointly (prophylactically or especially therapeutically) active. This means in particular that there is at least one beneficial effect, e.g. a mutual enhancing of the effect of the combination partners (i) and (ii), in particular a synergism, e.g. a more than additive effect, additional advantageous effects (e.g. a further therapeutic effect not found for any of the single compounds), less side effects, a combined therapeutic effect in a non-effective dosage of one or both of the combination partners (i) and (ii), and very preferably a clear synergism of the combination partners (i) and (ii).

For example, the term “jointly (therapeutically) active” may mean that the compounds may be given separately or sequentially (in a chronically staggered manner, especially a sequence-specific manner) in such time intervals that they preferably, in the warm-blooded animal, especially human, to be treated, and still show a (preferably synergistic) interaction (joint therapeutic effect). A joint therapeutic effect can, inter alia, be determined by following the blood levels, showing that both compounds are present in the blood of the human to be treated at least during certain time intervals, but this is not to exclude the case where the compounds are jointly active although they are not present in blood simultaneously.

The present invention thus pertains to a combination product for simultaneous, separate or sequential use, such as a combined preparation or a pharmaceutical fixed combination, or a combination of such preparation and combination.

In the combination therapies of the invention, the compounds useful according to the invention may be manufactured and/or formulated by the same or different manufacturers. Moreover, the combination partners may be brought together into a combination therapy: (i) prior to release of the combination product to physicians (e.g. in the case of a kit comprising the compound of the invention and the other therapeutic agent); (ii) by the physician themselves (or under the guidance of a physician) shortly before administration; (iii) in the patient themselves, e.g. during sequential administration of the compound of the invention and the other therapeutic agent.

In certain embodiments, any of the above methods involve further administering one or more other (e.g. third) co-agents, especially a chemotherapeutic agent.

Thus, the invention relates in a further embodiment to a combination product, particularly a pharmaceutical composition, comprising a therapeutically effective amount of (i) an FGFR tyrosine kinase inhibitor and (ii) a MET tyrosine kinase inhibitor, or a pharmaceutically acceptable salt thereof, respectively, and at least one third therapeutically active agent (co-agent), e.g. another compound (i) and/or (ii) or a different co-agent. The additional co-agent is preferably selected from the group consisting of an anti-cancer agent; and an anti-inflammatory agent.

Also in this case, the combination partners forming a corresponding product according to the invention may be mixed to form a fixed pharmaceutical composition or they may be administered separately or pairwise (i.e. before, simultaneously with or after the other drug substance(s)).

A combination product according to the invention can besides or in addition be administered especially for cancer therapy in combination with chemotherapy, radiotherapy, immunotherapy, surgical intervention, or a combination of these. Long-term therapy is equally possible as is adjuvant therapy in the context of other treatment strategies, as described above. Other possible treatments are therapy to maintain the patient's status after tumor regression, or even chemo-preventive therapy, for example in patients at risk.

Possible anti-cancer agents (e.g. for chemotherapy) as co-agents include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity; anti-angiogenic compounds; compounds which target, decrease or inhibit the activity of a protein or lipid phosphatase; gonadorelin agonists; anti-androgens; methionine aminopeptidase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors; kinesin spindle protein inhibitors; MEK inhibitors; leucovorin; EDG binders; antileukemia compounds; ribonucleotide reductase inhibittors; S-adenosylmethionine decarboxylase inhibitors; angiostatic steroids; corticosteroids; other chemotherapeutic compounds (as defined below); photosensitizing compounds.

Further, alternatively or in addition combination products according to the invention may be used in combination with other tumor treatment approaches, including surgery, ionizing radiation, photodynamic therapy, implants, e.g. with corticosteroids, hormones, or they may be used as radiosensitizers.

The term “a commercial package” as used herein defines especially a “kit of parts” in the sense that the components (a) MET tyrosine kinase inhibitor and (b) FGFR tyrosine kinase inhibitor as defined above and below, and optionally further co-agents, can be dosed independently or by use of different fixed combinations with distinguished amounts of the components (a) and (b), i.e., simultaneously or at different time points. Moreover, these terms comprise a commercial package comprising (especially combining) as active ingredients components (a) and (b), together with instructions for simultaneous, sequential (chronically staggered, in time-specific sequence) or separate use thereof in the delay of progression or treatment of a proliferative disease. The parts of the kit of parts can then, e.g., be administered simultaneously or chronologically staggered, that is at different time points and with equal or different time intervals for any part of the kit of parts. Very preferably, the time intervals are chosen such that the effect on the treated disease in the combined use of the parts is larger than the effect which would be obtained by use of only any one of the combination partners (a) and (b) (as can be determined according to standard methods. The ratio of the total amounts of the combination partner (a) to the combination partner (b) to be administered in the combined preparation can be varied, e.g., in order to cope with the needs of a patient sub-population to be treated or the needs of the single patient which different needs can be due to the particular disease, age, sex, body weight, etc. of the patients. Preferably, there is at least one beneficial effect, e.g., a mutual enhancing of the effect of the combination partners (a) and (b), in particular a more than additive effect, which hence could be achieved with lower doses of each of the combined drugs, respectively, than tolerable in the case of treatment with the individual drugs only without combination, producing additional advantageous effects, e.g., less side effects or a combined therapeutic effect in a non-effective dosage of one or both of the combination partners (components) (a) and (b), and very preferably a strong synergism of the combination partners (a) and (b).

Both in the case of the use of the combination of components (a) and (b) and of the com-mercial package, any combination of simultaneous, sequential and separate use is also possible, meaning that the components (a) and (b) may be administered at one time point simultaneously, followed by administration of only one component with lower host toxicity either chronically, e.g., more than 3-4 weeks of daily dosing, at a later time point and subsequently the other component or the combination of both components at a still later time point (in subsequent drug combination treatment courses for an optimal effect) or the like.

The combination products according to the present invention are appropriate for the treatment of various diseases that are mediated by, especially depend on, the activity of FGFR and/or MET tyrosine kinase, respectively. They can thus be used in the treatment of any of the diseases that can be treated by FGFR tyrosine kinase inhibitors and MET tyrosine kinase inhibitors.

The term “FGFR tyrosine kinase activity and/or MET tyrosine kinase activity mediated disease” refers especially to a disease in which activity of one or both kinases leads to abnormal activity of the regulatory pathways including one of both kinases, especially where one or both of the kinases is overactive, e.g. due to overexpression, mutation or relative lack of activity of other regulatory pathways in the cell, e.g. where there is amplification, constitutive activation and/or over-activation of preceding or subsequent regulatory elements.

FGFR inhibitors are e.g. useful in the treatment of one or more of the diseases which respond to an inhibition of FGFR activity, especially a neoplastic or tumor disease, especially solid tumor, more especially those cancers in which FGFR kinases are implicated including breast cancer, gastric cancer, lung cancer, cancer of the prostate, bladder cancer and endometrial cancer.

Further cancers include cancer of the kidney, liver, adrenal glands, stomach, ovaries, colon, rectum, pancreas, vagina or thyroid, sarcoma, glioblastomas and numerous tumours of the neck and head, as well as leukemias and multiple myeloma. FGFR inhibitors are also useful in the treatment of a warm-blooded animal having a disorder mediated by the fibroblast growth factor receptor, in particular 8p11 myeloproliferative syndrome (EMS), pituitary tumors, retinoblastoma, synovial sarcoma, chronic obstructive pulmonary disease (COPD), seborrheic keratosis, obesity, diabetes and related disorders, autosomal dominant hypophosphatemic Rickets (ADHR), X-chromosome linked hypophosphatemic rickets (XLH), tumor-induced osteomalacia (TIO) and fibrous dysplasia of the bone (FD) as well as to a method of promoting localized neochondrogenesis, as well as a method of treating hepatocellular carcinoma, lung cancer, especially pulmonary adenocarcinoma, oral squameous cell carcinoma or esophageal squameous cell carcinoma, or any combination of two or more such diseases.

MET inhibitors are e.g. useful in the treatment of MET related diseases, especially cancers that display evidence for simultaneous activation of MET and FGFR, including gene amplification, activating mutations, expression of cognate RTK ligands, phosphorylation of RTKs at residues indicative of activation, e.g. where the cancer is selected from the group consisting of brain cancer, stomach cancer, genital cancer, urinary cancer, prostate cancer, (urinary) bladder cancer (superficial and muscle invasive), breast cancer, cervical cancer, colon cancer, colorectal cancer, glioma (including glioblastoma, anaplastic astrocytoma, oligoastrocytoma, oligodendroglioma), esophageal cancer, gastric cancer, gastrointestinal cancer, liver cancer, hepatocellular carcinoma (HCC) including childhood HCC, head and neck cancer (including head and neck squamous-cell carcinoma, nasopharyngeal carcinoma), Hurthle cell carcinoma, epithelial cancer, skin cancer, melanoma (including malignant melanoma), mesothelioma, lymphoma, myeloma (including multiple myeloma), leukemias, lung cancer (including non-small cell lung cancer (including all histological subtypes: adenocarcinoma, squamous cell carcinoma, bronchoalveolar carcinoma, large-cell carcinoma, and adenosquamous mixed type), small-cell lung cancer), ovarian cancer, pancreatic cancer, prostate cancer, kidney cancer (including but not limited to papillary renal cell carcinoma), intestine cancer, renal cell cancer (including hereditary and sporadic papillary renal cell cancer, Type I and Type II, and clear cell renal cell cancer); sarcomas, in particular osteosarcomas, clear cell sarcomas, and soft tissue sarcomas (including alveolar and (e.g. embryonal) rhabdomyosarcomas, alveolar soft part sarcomas); thyroid carcinoma (papillary and other subtypes).

MET inhibitors are e.g. also useful in the treatment of cancer wherein the cancer is stomach, colon, liver, genital, urinary, melanoma, or prostate. In a particular embodiment, the cancer is liver or esophageal.

MET inhibitors are e.g. also useful in the treatment of colon cancer, including metastases, e.g. in the liver, and of non-small-cell lung carcinoma.

MET inhibitors are e.g. also may be used in the treatment of hereditary papillary renal carcinoma (Schmidt, L. et al. Nat. Genet. 16, 68-73, 1997) and other proliferative diseases in which c-MET is overexpressed or constitutively activated by mutations (Jeffers and Vande Woude. Oncogene 18, 5120-5125, 1999; and reference cited therein) or chromosomal rearrangements (e.g. TPR-MET; Cooper et al. Nature 311, 29-33, 1984; Park, et al. Cell 45, 895-904, 1986).

The combination product of the present invention is especially appropriate for treatment of any of the cancers mentioned above amenable to FGFR or Met inhibitor treatment, especially a cancer selected from adenocarcinoma (especially of the breast or more especially of the lung), rhabdomyosarcoma, osteosarcoma, urinary bladder carcinoma and glioma.

The term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective to (1) at least partially alleviating, inhibiting, preventing and/or ameliorating a condition, or a disorder or a disease (i) mediated by cMet and/or mediated by FGFR activity, or (ii) characterized by activity (normal or abnormal) of cMet and/or of FGFR; or (2) reducing or inhibiting the activity of cMet and/or of FGFR; or (3) reducing or inhibiting the expression of cMet and/or FGFR. In another non-limiting embodiment, the term “a therapeutically effective amount” refers to the amount of the compound of the present invention that, when administered to a cell, or a tissue, or a non-cellular biological material, or a medium, is effective to at least partially reducing or inhibiting the activity of cMet and/or FGFR; or at least partially reducing or inhibiting the expression of MET and/or FGFR.

As used herein, the term “subject” refers to an animal. Typically the animal is a mammal. A subject also refers to for example, primates (e.g., humans), cows, sheep, goats, horses, dogs, cats, rabbits, rats, mice, fish, birds and the like. In certain embodiments, the subject is a primate. In yet other embodiments, the subject is a human.

“And/or” means that each one or both or all of the components or features of a list are possible variants, especially two or more thereof in an alternative or cumulative way.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

The term “treatment” comprises, for example, the prophylactic or especially therapeutic administration of the combination partners to a warm-blooded animal, preferably to a human being, in need of such treatment with the aim to cure the disease or to have an effect on disease regression or on the delay of progression of a disease.

As used herein, a subject is “in need of” a treatment if such subject would benefit biologically, medically or in quality of life from such treatment.

As used herein, the term “a,” “an,” “the” and similar terms used in the context of the present invention (especially in the context of the claims) are to be construed to cover both the singular and plural unless otherwise indicated herein or clearly contradicted by the context.

The combinations according to the invention can be prepared in a manner known per se and are those suitable for enteral, such as oral or rectal, and parenteral administration to mammals (warm-blooded animals), including man, comprising a therapeutically effective amount of at least one pharmacologically active combination partner alone or in combination with one or more pharmaceutically acceptable carriers, especially suitable for enteral or parenteral application. In one embodiment of the invention, one or more of the active ingredients are administered orally.

As used herein, the term “carrier” or “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The pharmaceutical combination product according to the invention (as fixed combination, or as kit, e.g. as combination of a fixed combination and individual formulations for one or both combination partners oras kit of individual formulations of the combination partners) comprises the combination partners (at least one MET tyrosine kinase inhibitor, at least one FGFR tyrosine kinase inhibitor, and optionally one or more further co-agents) of the present invention and one or more pharmaceutically acceptable carrier materials (carriers, excipients). The combination products or the combination partners constituting it can be formulated for particular routes of administration such as oral administration, parenteral administration, and rectal administration, etc. In addition, the combination products of the present invention can be made up in a solid form (including without limitation capsules, tablets, pills, granules, powders or suppositories), or in a liquid form (including without limitation solutions, suspensions or emulsions). The combination products and/or their combination partners can be subjected to conventional pharmaceutical operations such as sterilization and/or can contain conventional inert diluents, lubricating agents, or buffering agents, as well as adjuvants, such as preservatives, stabilizers, wetting agents, emulsifers and buffers, etc.

In all formulations, the active ingredient(s) forming part of a combination product according to the present invention can be present each in a relative amount of 0.5 to 95% of weight of the corresponding formulation (regarding the formulation as such, that is without packaging and leaflet), e.g. from 1 to 90, 5 to 95, 10 to 98 or 10 to 60 or 40 to 80% by weight, respectively.

The pharmaceutical combination product of the present invention can e.g. be in unit dosage of about 1-1000 mg of active ingredient(s) for a subject of about 50-70 kg, or about 1-500 mg or about 1-250 mg or about 1-150 mg or about 0.5-100 mg, or about 1-50 mg, or 50 to 900, 60 to 850, 75 to 800 or 100 to 600 mg, respectively, of any one or in particular the sum of active ingredients. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

### DESCRIPTION OF THE FIGURES

FIG. 1: Primary Secretome Rescue of MKN-45 cells with cMET amplification and MET-dependent growth in the presence of the MET-inhibitor (E)-2-(1-(3-((7-fluoroquinolin-6-yl)methyl)-imidazo[1,2-b]pyridazin-6-yl)ethylidene)hydrazinecarboxamide (Cpd. A)

FIG. 2: Primary Secretome Rescue or RT-112 cells with FGFR3 gene amplification and FGFR3-dependent growth in the presence of the FGFR inhibitor 3-(2,6-dichloro-3,5-dimethoxy-phenyl)-1-{6-[4-(4-ethylpiperazin-1-yl)-phenylamino]-pyrimidin-4-yl}-1-methyl-urea monophosphate (BGJ398)

FIG. 3: Reversal of rescue with selective inhibitors in MET-dependent MKN-45 cells, showing that the MET inhibitor Cpd. B is active, as is the combination with BGJ398 (FGFR inhibitor), while BGJ398 and the (dual Erb2 and) EGFR inhibitor lapatinib are not sufficient. Cpd. B=2-fluoro-N-methyl-4-[(7-quinolin-6-yl-methyl)-imidazo[1,2-b]triazin-2-yl]benzamide (MET inhibitor), FGF7=Fibroblast Growth Factor 7 (activator of FGFR), Lapatinib=(ErbB2 and) EGFR inhibitor, NRG1=Neuregulin 1 (increases ERBB2 tyrosine phosphorylation).

FIG. 4: Reversal of rescue with selective inhibitors in MET-dependent MKN-45 cells, showing that the MET inhibitor Cpd. A is active, as is the combination with BGJ398 (FGFR inhibitor), while BGJ398 and the (dual Erb2 and) EGFR inhibitor lapatinib are not sufficient. FGF73=Fibroblast Growth Factor 7 (activator of FGFR), Lapatinib=(ErbB2 and) EGFR inhibitor, NRG1=Neuregulin 1 (EGFR-Aktivator).

FIG. 5: Reversal of Rescue with selective inhibitors in EGFR-dependent RT-112 cells, showing that the EGFR-inhibitor BGJ398 is active, as is the combination with Cpd. B, while Cpd. B alone as well as the (dual ErbB2 and) EGFR inhibitor lapatinib are not sufficient. HGF and NRG1 are as defined for FIG. 3.

FIG. 6: Reversal of Rescue with selective inhibitors in EGFR-dependent RT-112 cells, showing that the EGFR-inhibitor BGJ398 is active, as is the combination with Cpd. A, while Cpd. A alone as well as the (dual ErbB2 and) EGFR inhibitor lapatinib are not sufficient. HGF and NRG1 are as defined for FIG. 3.

FIG. 7: Showing of Synergy (areas marked with a solid frame indicate synergy) for different combinations:
A) Cpd. B and BGJ398 in KYM-1 monolayer culture;
B) Cpd. B and BGJ298 in KYM-1 nonadherent culture;
C) Cpd. B and BGJ398 in KYM-1 soft agar culture;
D) Cpd. B and BGJ298 in MG-63 monolayer culture;
E) Cpd. B and BGJ298 in M-63 soft agar culture;
F) Cpd. B and BGJ398 in Hs 683 monolayer culture.

FIG. 8: Showing of synergy in terms of summed-up effective level in percent relative to drug self combination based in the Loewe model. Compound concentrations in micromolar, layout as in FIG. 7.

FIG. 9: Anti-tumor activity of an FGFR and MET inhibitor combination in a primary lung cancer xenograft model. (A) Tumor growth curves in cohorts of tumor-bearing mice treated with the indicated regimens. The arrow marks a reduction in the frequency of Cpd. B dosing from twice to once daily. 1=vehicle control (circle), 2=10 mg/kg Cpd. B bid/qd, 3=40 mg/kg BGK398 qd, 4=combination (B) Analysis of MET phosphorylation by ELISA in tumors 2 h or 12 h after the last Cpd. B dose. 1=vehicle control (circle), 0=10 mg/kg Cpd. B bid, 3=40 mg/kg BGJ398 qd, 4=combination.

The description of the figures is also part of the invention disclosure.

### EXAMPLES

The following Examples serve to illustrate the invention and provide specific embodiments, however they do not limit the scope of the invention:

BCA protein assay=assay based on biuret reaction (reduction of Cu(II) to Cu(I) cations by proteins in alkaline solution with bicinchonic acid as chromogenic agent that chelates the reduced copper and thus produces a purple complex with strong absorbance at 562 nm)

ECL=Enhanced Chemiluminescence (emission of light during the horse radish peroxidase snd hydrogen peroxide catalyzed oxidation of luminol)

### Cell Culture and Reagents

The MET-dependent adenocarcinoma line MKN-45, KYM-1 rhabdomyosarcoma line and MG-63 osteosarcoma lines were obtained from Health Science Research Resources Bank (Japan Health Sciences Foundation). The FGFR-dependent urinary bladder carcinoma RT-112 was obtained from Leibniz-lnstitut Deutsche Sammlung von Mikroorganismen und Zellkulturen. Hs-683 glioma line and HEK 293T/17 cells, a human kidney line expressing SV40 large T antigen, were purchased from American Type Culture Collection. Cpd. A, Cpd. B and BGJ398 were synthesized internally at Novartis.

### Creation of the Secretome Library

A bioinformatics pipeline was built to identify secreted and single pass transmembrane proteins; similar to previously described methods (Gonzalez, R., et al., PNAS, 2010; Lin, H., et al., Science, 2008). In brief, all human RefSeq protein sequences (June 2004 version containing 27,959 proteins) were filtered through the databases SWISSPROT and INTERPRO for previous annotation as secreted or transmembrane (Hunter, S., et al., Nucleic Acids Res, 2009; O'Donovan, C, et al., Brief Bioinform, 2002). Then protein sequences were analyzed with algorithms that identify signal sequences and transmembrane helices: TMHMM, SIGNALP, and PHOBIUS (Bendtsen, J. D., et al., J Mol Biol, 2004; Kail, L, et al., J Mol Biol, 2004; Krogh, A., et al., J Mol Biol, 2001). 2,803 unique gene IDs were selected and mapped to 3,432 clones; all were purchased from the he Invitrogen Ultimate ORF collection and DNA isolated using standard techniques. pcDNA-DEST40 was the plasmid vector for all clones and all clone inserts were confirmed by full sequencing.

### Example A

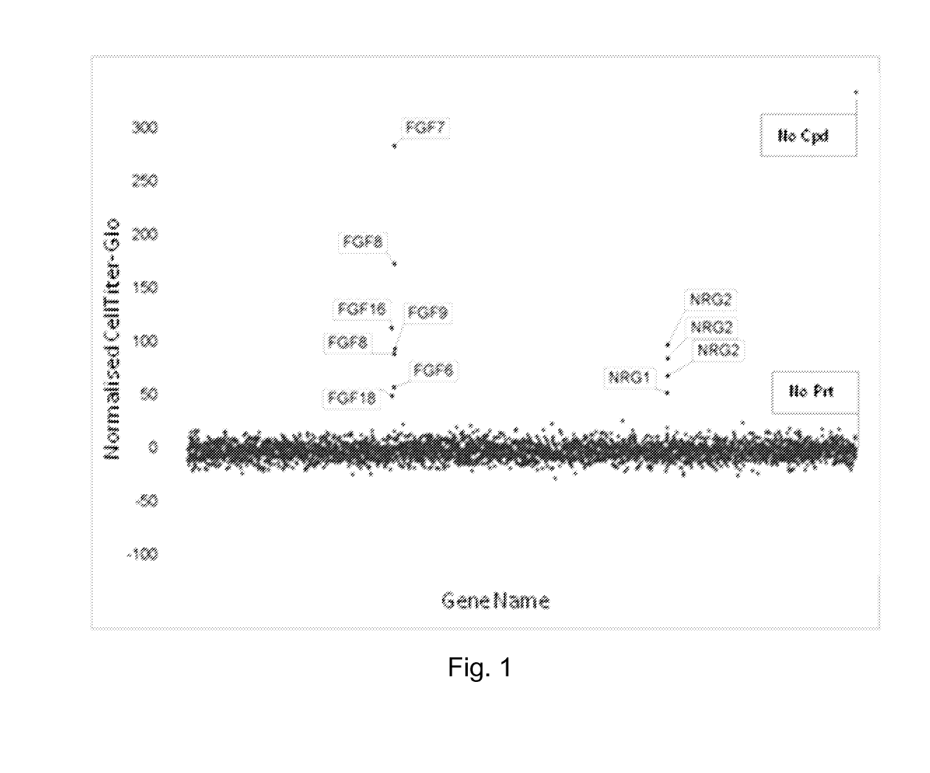

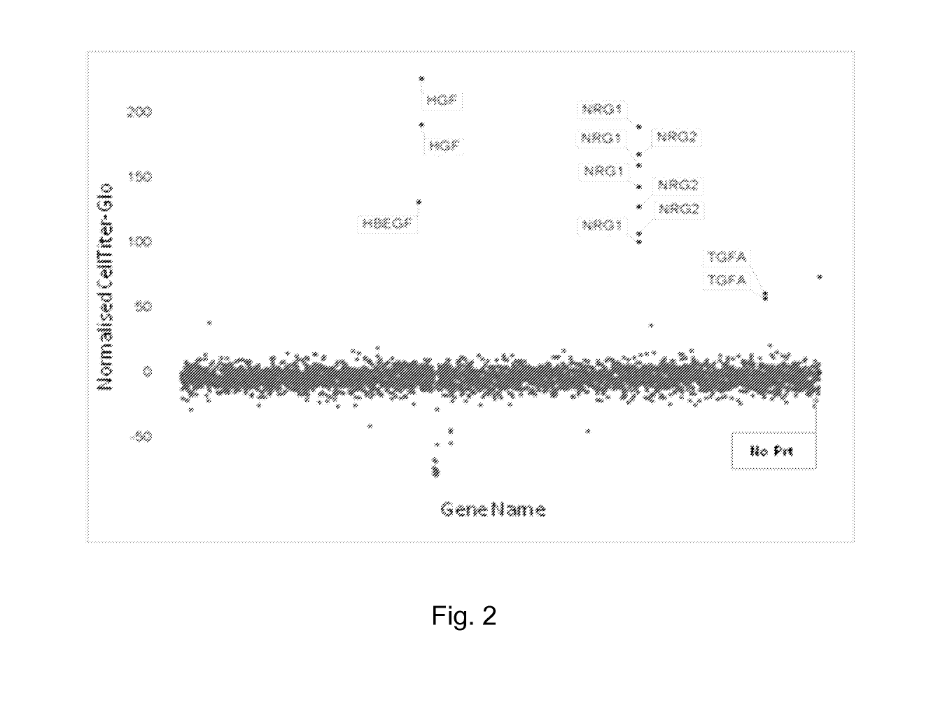

### Secretomics Screening (FIG. 1 and FIG. 2)

Supernatant production: DNA from the aforementioned clones was stamped into clear, tissue-culture treated 384-well plates, 4 μl/well at 7.5 ng/ul. Stamps were stored frozen at −20 C. until use. On day of experiment stamped cDNA plates were thawed and equilibrated to room temperature. HEK293T/17 cells were reverse-transfected as follows: Fugene HDwas diluted in Optimem to achieve a final ratio of 4:1 (nl Fugene HD:ng DNA). Diluted transfection reagent was added to stamped DNA plates 10 μl/well and allowed to incubate 30 minutes at room temperature. HEK293T/17 cells were then added at 7,000 cells/50 μl/well and incubated four days under standard tissue culture conditions to allow accumulation of secreted proteins in the media supernatant.

MKN-45 Secretome screen: MKN-45 cells were plated in white, tissue-culture treated 384-well plates (Greiner) at 3000 cells/20 μl DMEM +10% FBS/well and allowed to attach overnight. Supernatant from library-transfected HEK293T/17 cells was then transferred to the MKN-45 cells at 30 μl/well using a Biomek FX liquid handler (Beckman Coulter) with pipetting speeds reduced to minimize disturbance of the HEK293T/17 monolayer. As positive controls, the purified proteins rhEGF and rhNRG1-31 (R&D Systems) were added to isolated wells on each plate for a final concentration of 150 ng/ml; supernatant from mock-transfected HEK293T/17 wells were transferred as neutral controls. Following addition of supernatants and purified protein controls, Cpd. A diluted in DMEM was added at 10 μl/well for a final assay concentration of 100 nM. After 96 hours incubation, growth was measured using the CellTiter-Glo luminescent cell viability assay system (Promega). In brief, 30 μl CellTiter-Glo reagent was added to all wells, then incubated for 15 minutes at room temperature before reading luminescence on a Viewlux plate reader (Perkin Elmer). (FIG. 1)

RT-112 Secretome screen: The basic format was identical to the MKN-45 secretome screen with slight modifications. RT-112 cells were plated in EMEM+10% in white, tissue-culture treated 384-well plates at 1000 cells/20 μl/well and allowed to attach overnight. Supernatant from library-transfected HEK293T/17 cells was transferred as described above for the MKN-45 secretome screen. Purified proteins rhNRG1-31 and rhTGFα were added as positive controls, at a final concentration of 150 ng/ml. Following addition of supernatants and purified protein controls, BGJ398 diluted in DMEM was added at 10 μl/well for a final assay concentration of 100 nM. Cell viability was measured after 72 hours using CellTiter-Glo as described above. (FIG. 2)

In both screens assay data was normalized to vector only controls using the formula:

$$Normalized activity = 100 \times ( \frac{X - {vector}_{median}}{{vector}_{median}} )$$

where X is the raw value and vector_{median} is the median of vector control wells for a given plate.

Purified protein confirmation: The assay format for purified protein confirmation was identical to the format used for primary screening, with the exception that purified proteins were added at 30μl/well in place of HEK293T/17 supernatant, for a final concentration of 100 ng/ml.

### Example B

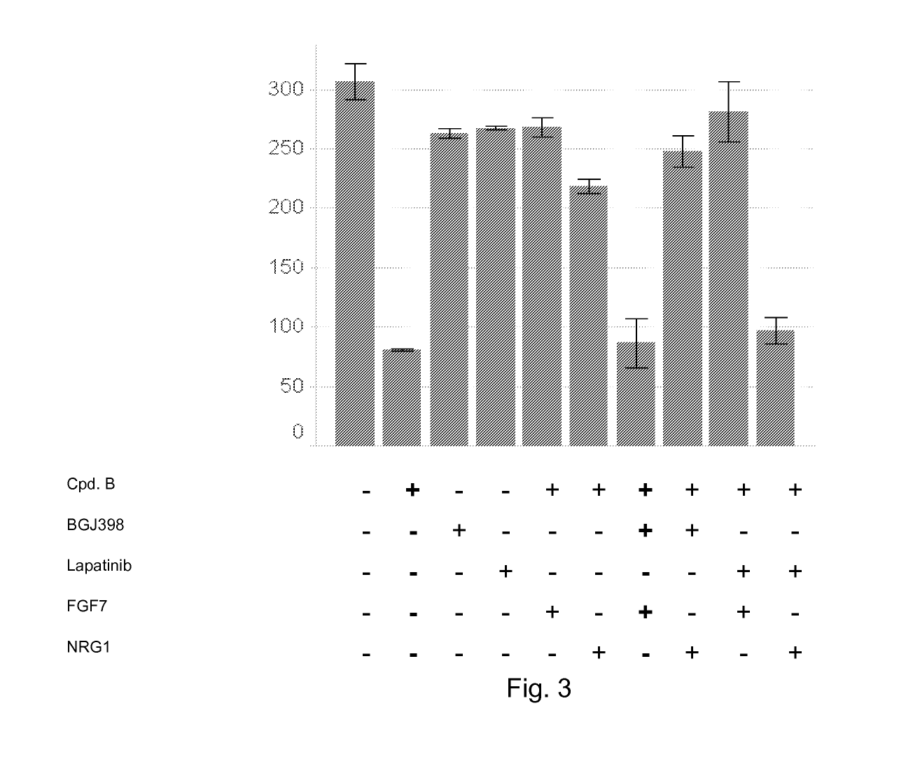

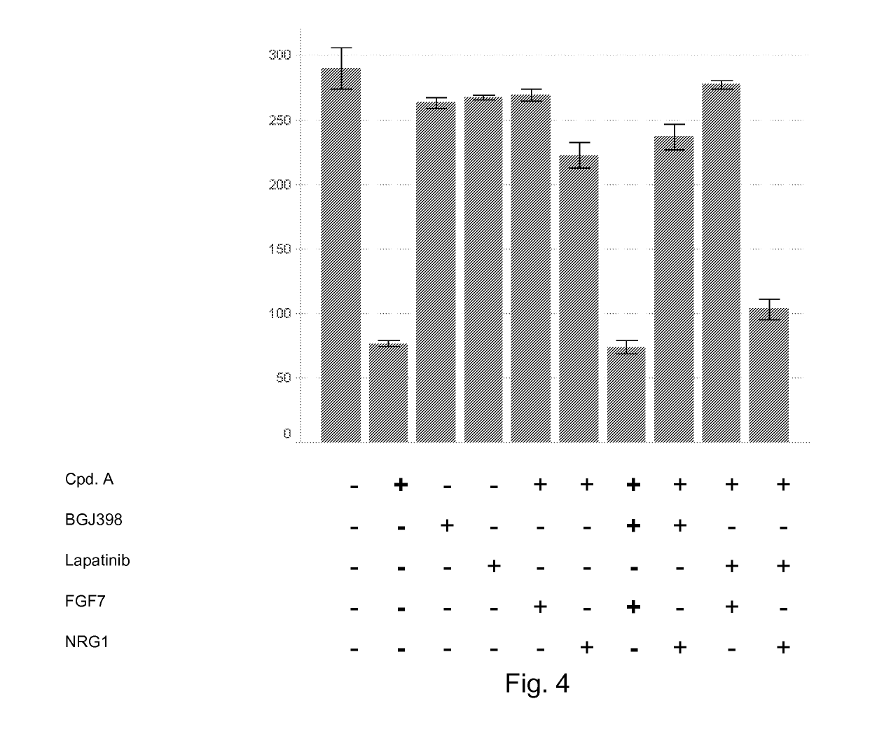

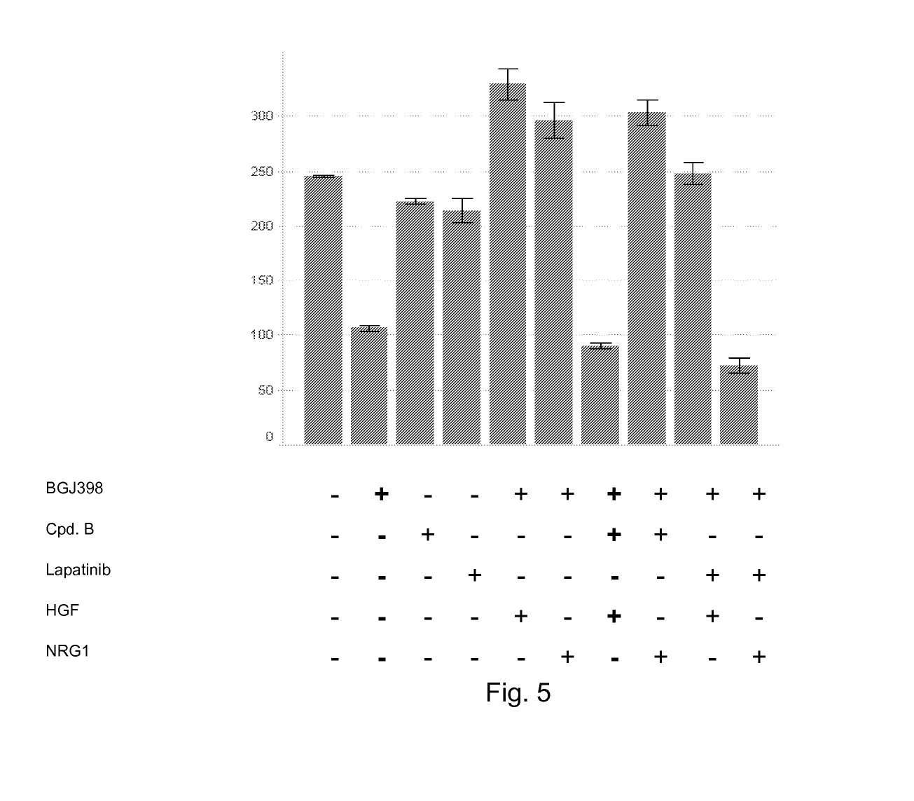

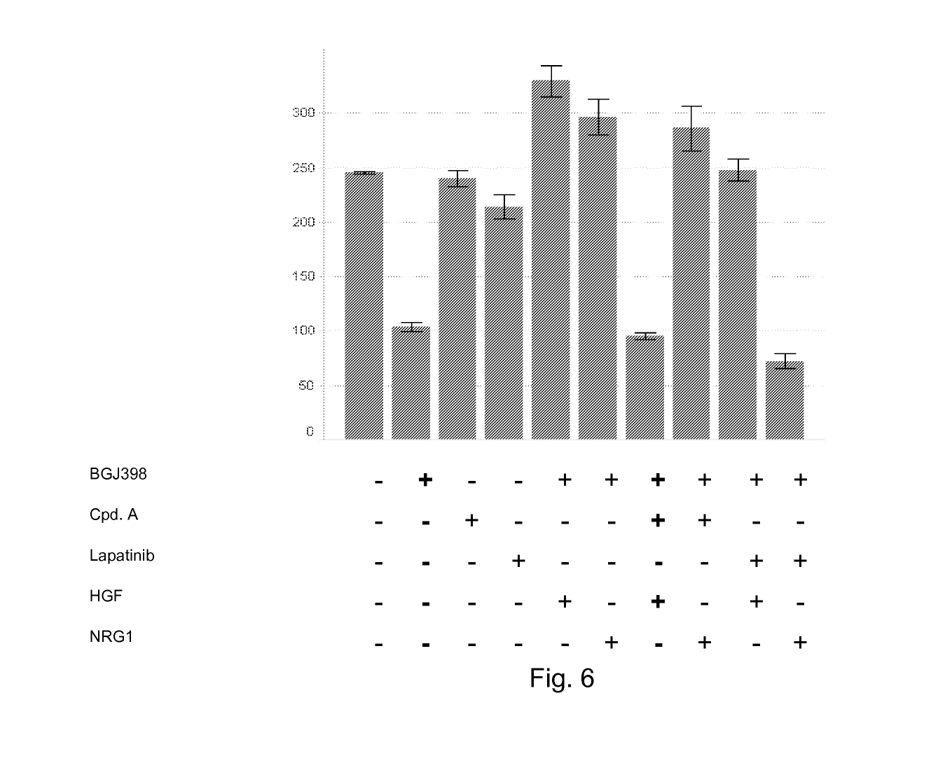

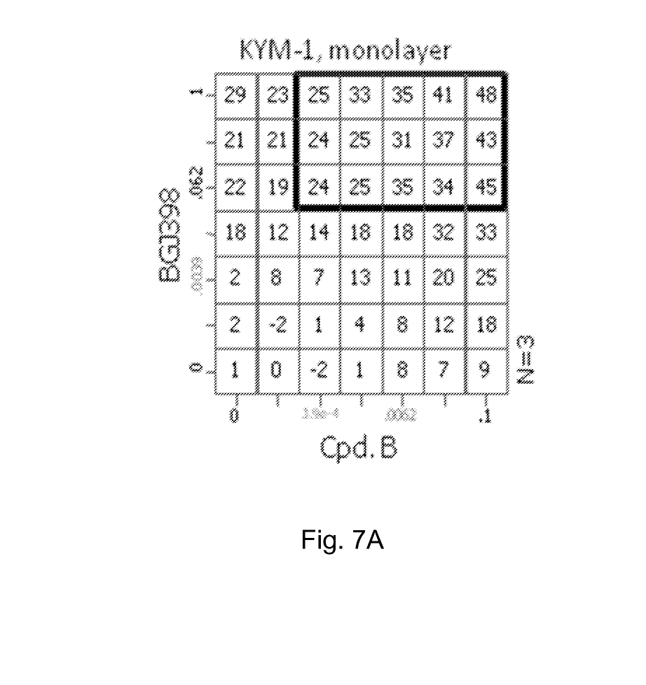

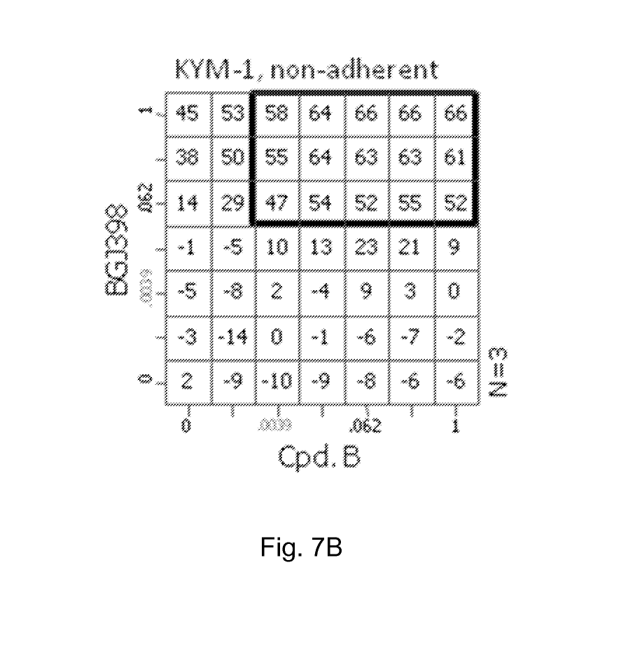

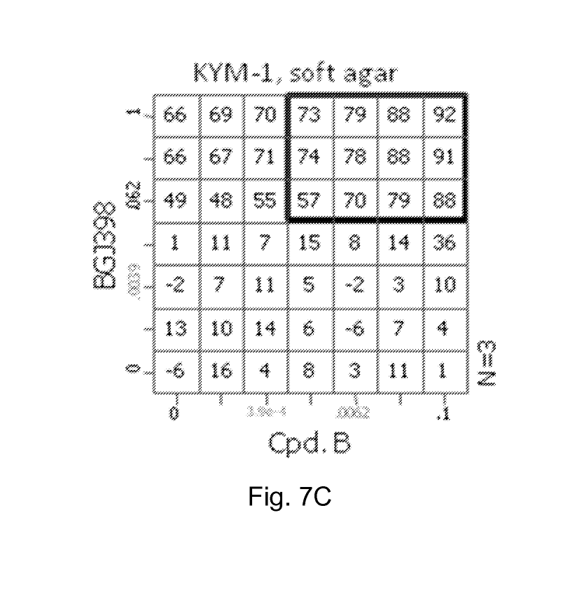

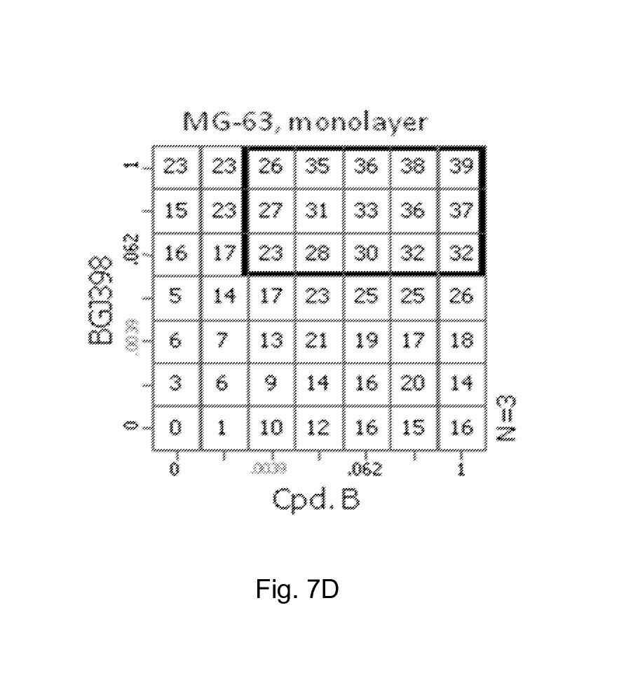

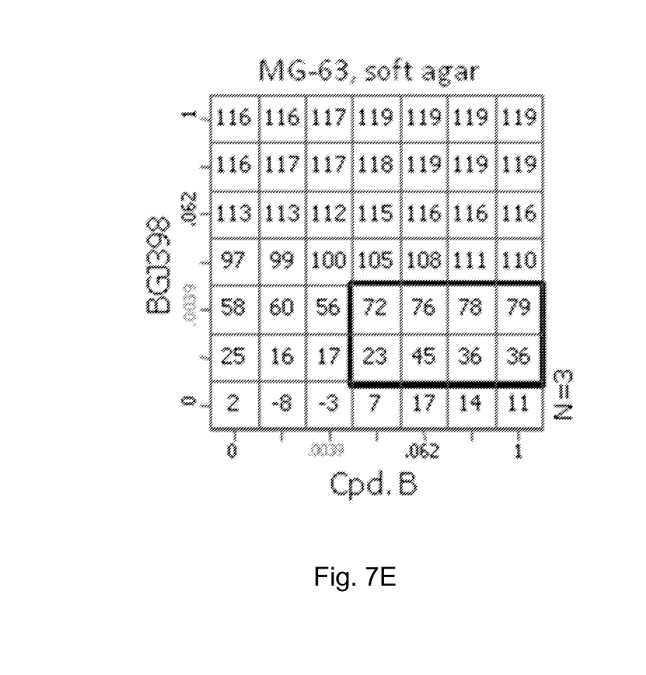

Reversal of Rescue with Selective Inhibitors (FIG. 3, FIG. 4, FIG. 5, FIG. 6))

MKN-45 Dual Inhibition: MKN-45 cells were seeded at 3000 cells/20 μl/well in 384-well plates and incubated overnight. Solutions of rhFGF7 and rhNRG-1 were prepared in DMEM+10%FBS, then added at 30 μl/well to achieve a final concentration of 250 ng/ml (one purified protein per treatment). The following single and dual inhibition treatments were prepared in DMEM and added at 10 nL/well: Cpd. A, Cpd. B, BGJ398, Lapatinib, Cpd. A and BGJ398, Cpd. B and BGJ398, Cpd. A and Lapatinib, Cpd. B and Lapatinib. Final concentrations for each compound, whether single or combined, were as follows: 100 nM for Cpd. A and Cpd. B, 500 nM for BGJ398 and 1.5 μM for Lapatinib. After 96 hours cell viability was measured by CellTiter-Glo as previously described. (FIG. 3, FIG. 4)

RT-112 Dual Inhibition: The format for the RT-112 dual inhibition experiment was similar to that described for MKN-45 with the following modifications. RT-112 cells were plated at 1000 cells/20 μl/well. Solutions of rhHGF and rhNRG-1 were added to achieve a final concentration of 250 ng/ml. Single and dual inhibition conditions were prepared as follows: BGJ398, Cpd. A, Cpd. B, Lapatinib, BGJ398 and Cpd. A, BGJ398 and Cpd. B, BGJ398 and Lapatinib. Final concentrations for each compound, whether single or combined, were as follows: 100nM for BGJ398, 500 nM for Cpd. A and Cpd. B, and 1.5 μM for Lapatinib. After 96 hours cell viability was measured by CellTiter-Glo as previously described. (FIG. 5, FIG. 6).

Western Blots (Figures not Shown) MKN-45 and RT-112 cells were treated in 6-well plates for 2 hours and 18 hours in the presence or absence of purified protein (rhFGF7, rhNRG1-31, or rhHGF), and/or inhibitor (Cpd. B, Cpd. A, BGJ398). Following wash with ice-cold PBS, cells were lysed with RIPA buffer (Thermo) containing phosphatase (Thermo) and proteinase (Roche) inhibitor cocktails. Total protein was quantified by bicinchoninic protein assay (Pierce). Aliquots of 20 μg were resolved by electrophoresis on NuPAGE SDS-PAGE 4-12% BIS-Tris gels before transfer to nitrocellulose membranes. Membranes were blocked for one hour at room temperature before overnight incubation at 4° C. with the following primary antibodies (source rabbit, 1:1000 final dilution): anti-phospho-Akt (Ser473), anti-phospho-MET (Tyr1234/1235), anti-phospho-MAPK/ERK(1/2) (Thr202/Tyr204), anti-AKT, anti-MAPK/Erk(1/2), and anti-α/β-Tubulin . Anti-MET was probed at 1:800 final dilution An internal antibody was used for phospho-FRS2(Y346) (1:1500 dilution). Membranes were then washed 3 times in PBS+0.1% Tween before addition of secondary antibody, IRDye 680LT goat anti-rabbit IgG , dilution 1:15000. After one hour at room temperature, membranes were washed and bands were visualized using an Odyssey Infrared Imager.

Results and Discussion: As we had no proof that active proteins were produced in sufficient quantities by cDNA transfection, we sought to orthogonally validate that the rescue effects observed during the screen were indeed mediated by the expected secreted proteins. To this end, we tested recombinant proteins from commercial sources in the same cell proliferation assay. We initially tested the potential of a palette of recombinant FGFs as well as of EGF and NRG1-3 to rescue MKN-45 in presence of the MET inhibitor Cpd. B (data not shown). Rescue could be confirmed with several FGF and EGF family members. As an additional validation step, we sought to demonstrate that the ligand effects were mediated by activation of their cognate RTKs. To this end, we used specific inhibitors—BGJ398 for FGFR1/2/3 and lapatinib for HER1/2—to reverse rescue (FIGS. 3, 4). These experiments were done both with Cpd. A (FIG. 4) and the equally selective MET inhibitor Cpd. B (FIG. 3). As expected, BGJ398 could selectively reverse rescue mediated by FGF7 while lapatinib reversed rescue by NRG1. To investigate whether common downstream signals are underlying the observed rescue effects, we analyzed the consequences of MET inhibition, ligand-mediated rescue, and inhibitor-mediated reversal on the level of protein phosphorylation by Western blotting. In the absence of added ligands only Cpd. A and Cpd. B, but not BGJ398 or lapatinib, had a profound effect on phosphorylation of several proteins (MET, ERK1/2, AKT, FRS2) in the MET-amplified cell line MKN-45. Addition of FGF7 re-activated FRS2 phosphorylation, a specific marker downstream of FGFR, and at the same time partially re-activated ERK1/2 phosphorylation with with no or minimal effect on AKT phosphorylation. This rescue effect could be reversed by BGJ398 but not lapatinib. NRG1 evoked similar effects with a more pronounced phospho-AKT re-activation, which were specifically sensitive to lapatinib. In summary, the observed effects on protein phosphorylation are in line with the effects on cell proliferation. Re-activation of ERK phosphorylation is more consistently re-activated by both FGF7 and NRG1 and hence appears to play a dominant role in sustaining cell growth in this cell line. We then turned to the originally FGFR-dependent cancer cell line RT-112 and conducted the analogous validation experiments. Recombinant HER ligands as well as HGF were found to reproduce the rescue effects observed in secretome screening. Furthermore, selective inhibition of the cognate RTKs could reverse rescue mediated by HGF or NRG1 (FIGS. 5, 6). Finally, effects on phosphorylation of selected proteins were assessed by Western blot. In RT-112 cells we did not observe basal AKT phosphorylation. In the absence of ligands, Cpd. A and B specifically inhibited MET phosphorylation, while BGJ398 reduced both FRS2 and ERK phosphorylation. Addition of HGF restored ERK phosphorylation in the presence of BGJ398, while simultaneous addition of Cpd. A or B prevented this rescue effect. Likewise, NGR1 caused lapatinib-sensitive restoration of ERK phosphorylation and also stimulated phosphorylation of AKT. Again, phosphorylation of ERK appeared to be better correlated with cell growth then AKT phosphorylation.

Together these results suggest that pair wise combination of HER, MET, and FGFR inhibitors may be necessary for therapeutic efficacy if two of these RTKs are activated simultaneously by either genetic alterations or by cognate ligands. While in the experiments described so far ligands were added from exogenous sources, ligands in cancer patients may originate from the tumor itself (autocrine stimulation) or from other sources, e.g. tumor-associated stroma (paracrine stimulation).

### Example C

### Combination Assays (see FIG. 7)

Combinatorial anti-proliferative effects of Cpd. B (“B) in the tables below) and BGJ398 (“C” in the tables below) were measured in KYM-1, MG-63, and Hs-683 cells on 96-well-plates using the concentration matrix depicted below:

| | 1 | 2 | 3 | 4 | 5 | 6 | 7 | 8 | 9 | 10 | 11 | 12 |
|---|---|---|---|---|---|---|---|---|---|---|---|---|
| a | — | — | — | — | — | — | — | — | — | — | — | — |
| b | — | DMSO | C1 | C1 + B6 | C1 + B5 | C1 + B4 | C1 + B3 | C1 + B2 | C1 + B1 | B1 | — | — |
| c | — | DMSO | C2 | C2 + B6 | C2 + B5 | C2 + B4 | C2 + B3 | C2 + B2 | C2 + B1 | B2 | — | — |
| d | — | DMSO | C3 | C3 + B6 | C3 + B5 | C3 + B4 | C3 + B3 | C3 + B2 | C3 + B1 | B3 | — | — |
| e | — | DMSO | C4 | C4 + B6 | C4 + B5 | C4 + B4 | C4 + B3 | C4 + B2 | C4 + B1 | B4 | — | — |
| f | — | DMSO | C5 | C5 + B6 | C5 + B5 | C5 + B4 | C5 + B3 | C5 + B2 | C5 + B1 | B5 | — | — |
| g | — | DMSO | C6 | C6 + B6 | C6 + B5 | C6 + B4 | C6 + B3 | C6 + B2 | C6 + B1 | B6 | — | — |
| h | — | — | — | — | — | — | — | — | — | — | — | — |

Concentrations used:

| Cpd. B | μM | BGJ398 |
|---|---|---|
| B1 | 1.0000 | C1 |
| B2 | 0.2500 | C2 |
| B3 | 0.0625 | C3 |
| B4 | 0.0155 | C4 |
| B5 | 0.0039 | C5 |
| B6 | 0.0010 | C6 |

Wells labeled with hyphens were filled up with the according volume of growth medium. In some experiments, the maximal concentration of Cpd. A was 10-fold lower, but the dilutions steps were kept as above.

Cells were grown under three different conditions (see FIG. 7): Experiments labeled “monolayer” were conducted on regular tissue culture plates, allowing cells to adhere and eventually form a monolayer. Cells were seeded on 3 plates per experiment (triplicates) in standard growth media as described above at a density of 5000 per well. Six to eight wells on a separate plate were seeded to quantify the amount of viable cells at the point of compound addition. 24 h later, separate dilution series for each compound were prepared in growth medium at 10-fold of the final concentration starting from 10 mM DMSO stocks. DMSO-only controls were included as indicated. Aliquots of 10 μL for each compound dilution were added according to the matrix shown above, resulting in a final volume of 100 μL. At the same time, viable cells on the separate plate mentioned above were quantified using a resazurin sodium salt dye reduction readout. Specifically, 10 mL of a 0.13 mg/mL stock were added per well and plates were incubated for 2 h in a cell culture incubator before measuring absorptions (excitation 560 nm, emission 590 nm). The compound-treated cells were incubated for 72 hours followed by a resazurin assay as above. Percent inhibition was calculated by (a) subtracting the readout of seeded cells at the time of compound addition and (b) setting DMSO-only treated cells to 0% inhibition and the readout of seeded cells to 100% inhibition. Values above 100% are thus suggesting cell death over the course of incubation with compound. Quantification of Synergy was done using the methods described in G. R. Zimmermann et al., Drug Discovery Today, Vol. 12, No. ½, 2007, pages 34 to 42, and especially J. Lehár et al., Nature Biotechnology Vol. 27, No. 7, 2009, pages 659 to 666, in short by iteratively calculating the Loewe additive response ILoewe (X, Y) at each dose matrix point from the single-agent response curves, and then summing the differences between ILoewe and the experimental data. Where the sum was larger than that from mere addition of the ILoewe data, synergy was given (X, Y are the drug concentrations of drug X and drug Y on the X and Y axis, respectively).

The experiment in KYM-1 cells labeled as “non-adherent” was conducted in the same way except that Costar® ultra low adherent 96-well-plates were used. On these plates cells were not able to attach to plastic and grew in two-dimensional aggregates. The other two cell lines did not grow under these conditions.

For experiments labeled “soft agar” the cells were embedded in semi-solid media to allow three-dimensional aggregate formation. Specifically, agarose Type VII was dissolved in PBS at a concentration of 2.7%. The solution was then kept at 50° C. until immediately before plating and diluted with a 2-fold volume of cell line-specific growth medium as described above. Diluted agarose was then mixed with a 2-fold volume containing the respective cells and aliquots of 150 μL containing 3000 cells were quickly distributed on Costar ultra low adherent 96-well-plates. The final agarose concentration was thus 0.3%. Again, wells on a separate plate were seeded to quantify the amount of cells at the point of compound addition. After 24 h, compound dilution series were prepared so that a overlay of a total of 80 nL of diluted compounds in growth media would result in the final concentrations indicated in the scheme above, resulting in a total volume of 230 μL. Seeded cells were quantified by addition of 20 nL resazurin solution and incubation for 5 h. Compound-treated cells were incubated for 7 to 10 days and colonies were quantified with resazurin. Percent inhibition and synergy were calculated as above.

### Western Blots (Data Not Shown)

The indicated cell lines were seeded on 6-well-plates at a density of 500000 cells/well, left for 24 h to attach and then treated with a final concentration of 1 nM of the indicated compounds for another 24 h. Growth media were then removed, cells were washed twice with ice-cold PBS and lysed in 50 mmol/L Tris pH 7.5, 120 mmol/L NaCl, 20 mmol/L NaF, 1 mmol/L EDTA, 6 mmol/L EGTA, 1 mmol/L Benzamidin, 0.2 mmol/L PMSF, 100 mmol/L sodium vanadate, 1% NP-40. The protein concentration of cleared lysates was determined with the BCA Protein Assay Kit. 80 μg protein of each sample was separated by SDS-PAGE on NuPage 4-12% Tris-Bis Midi gels, transferred to a PVDF-membrane, and probed with antibodies as listed above. After washing and incubation with secondary HRP-linked antibodies, bands were visualized using ECL detection reagent.

The results are: Gene expression patterns suggest autocrine activation of MET and FGFR1.

Simultaneous activity of two RTKs could be the cause of primary or acquired resistance to selective kinase inhibitors. In order to investigate whether co-activation of RTKs through autocrine loops or by other genetic alterations is found in established cancer cell lines and whether this modulates the response to selective RTK inhibitors, we analyzed available expression and copy number profiles from a large set of cancer cell lines named the Broad-Novartis Cancer Cell Line Encyclopedia (http://www.broadinstitute.org/ccle/home). We focused our analysis on MET because of the relative simplicity—one receptor, one ligand—as well as on the FGFR family due to the novel discovery of cross-talk with MET. Since high level amplification of MET (as in MKN-45) and any of the FGFRs (as in RT-112) was found to be mutually exclusive, we turned our attention to potential autocrine loops. By applying a simple rank-order algorithm using expression values for MET, HGF, FGFRs, and FGFs, we identified cell lines with potential for dual autocrine RTK activation. Three promising and experimentally tractable candidates were then selected for combination studies (FIG. 7). The KYM-1 rhabdomyosarcoma cell line was found to express high MET and HGF levels as well as high FGFR1 and FGF20. We tested these cells in vitro in several experimental settings: First, we grew cells on adherent plates in monolayer. As a next step, we employed non-adherent plates, either in the presence or absence of semi-solid media. Under these conditions, especially in semi-solid media, cells grew in a more clustered manner, potentially supporting autocrine stimulation and resembling more closely a tumor in vivo. We incubated cells with combinations of Cpd. B and BGJ398 in several concentrations using a “checkerboard” layout (FIG. 7). While MET inhibition on its own had no effect, FGFR inhibition led to partial growth suppression. Importantly, combined inhibition of both RTKs suppressed growth more profoundly than either single agent. This finding was more pronounced when cells grew in clusters and supports the hypothesis that co-activation of RTKs can alleviate the dependence on a single RTK. In the osteosarcoma cell line MG-63 (high MET/HGF, high FGFR1/FGF18) we observed a similar combination effect in monolayer proliferation assays. Unexpectedly, in semi-solid media strong growth inhibition with BGJ398 alone was observed and combination with Cpd. B was rather beneficial at low concentrations of BGJ398 where inhibition of FGFR1 may be incomplete. While this result still argues for simultaneous activity of FGFR1 and MET in MG-63 cells, FGFR1 appears to be the dominant driver for growth. Lastly, we tested the glioma cell line Hs 683 (high MET/HGF, high FGFR1/FGF7) and observed clearly enhance growth inhibition in monolayer assays when simultaneously inhibiting both RTKs (FIG. 7F). We found that each compound as single agent effectively inhibited its own target, but effects on the downstream signal transducers AKT and MAPK were less apparent. Importantly, combinatorial effects were apparent in each cell line on the level of ERK phosphorylation inhibition, while AKT phosphorylation was unaffected. When performing the same analysis in KYM-1 cells grown in non-adherent plates, we observed a much more pronounced combination effect on ERK phosphorylation and a modest reduction in AKT phosphorylation with drug combination (data not shown).

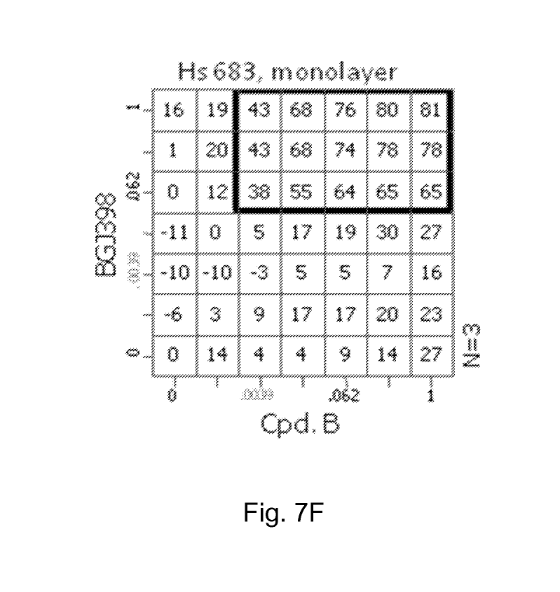

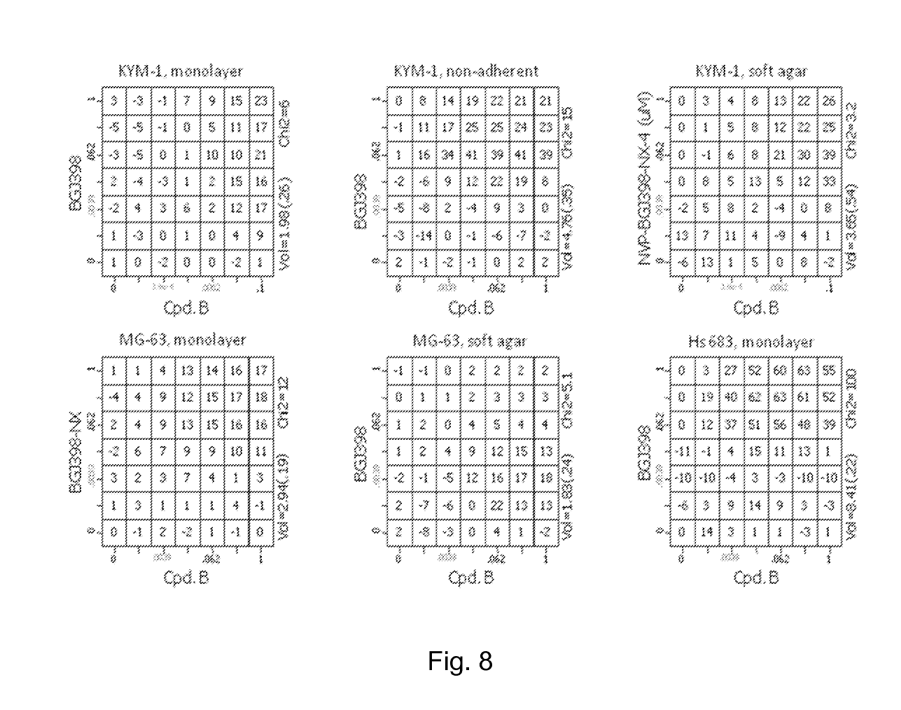

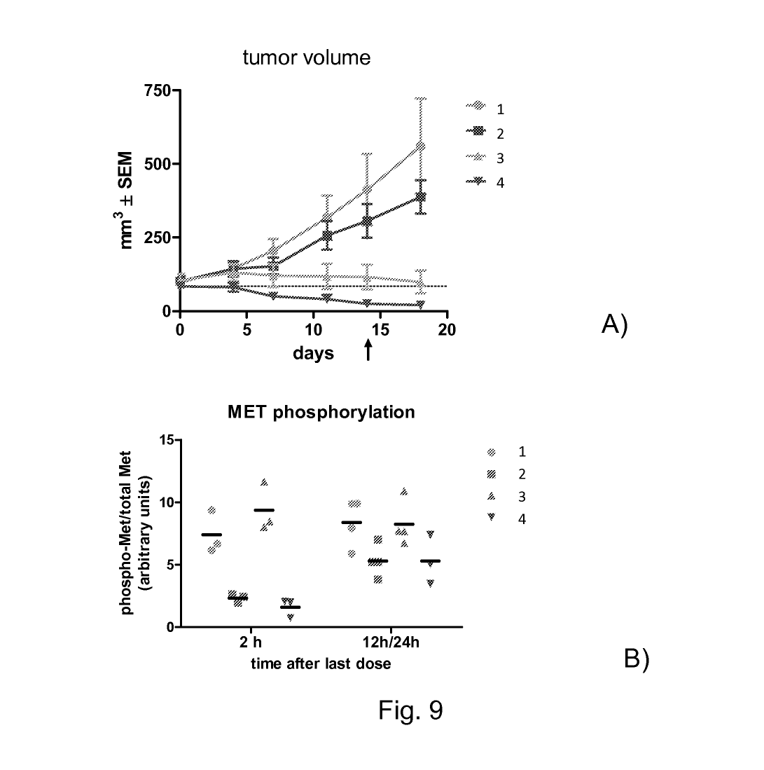

FIG. 8 shows the additional (or reduced) effect level in percent relative to drug self combination based on the Loewe model. The compound concentrations are in micromolar. The layout is as described for FIG. 7.

### Example D

In vivo Mouse Study (FIG. 8)

The in vivo anti-tumor efficacy study in the patient-derived non-small cell lung cancer xenograft LXFL 1121 was carried out. Xenografts were grown subcutaneously in nude mice. Eight tumor-bearing mice each were treated with either vehicle control, Cpd. B alone, BGJ398 alone or both drugs in combination at the doses and frequencies indicated. Note that frequency of Cpd. B administration was reduced from twice to once daily at study day 14 after body weight loss in the combination group was observed. In the combination group, one animal died after study day 7 and one after study day 18 for unknown reasons. Tumor volumes were measured on the indicated days and synergy of the combination treatment was assessed by the method of Clarke, R., Breast Cancer Res. Treat., 41997.

For pharmacokinetic/pharmacodynamic analysis, animals of the vehicle and Cpd. B-only groups were sacrificed after study day 21, half of the animals each at 2 h or 12 h after last administration. Plasma and tumor samples were collected. Treatment in the two remaining groups was stopped after day 21 and tumors were allowed to re-grow in order to obtain sufficient material for pharmacodynamic analysis. On day 61, a final dose of BGJ398 or BGJ398/Cpd. B combination were given and half of the remaining mice were sacrificed after 2 h. A second dose of Cpd. B was administered to the combination group 12 h after the first, and the remaining animals were sacrified 24 h after BGJ398=12 h after the last Cpd. B administration.

Snap-frozen tumor samples were pulverized by hand in a steel mortar that was cooled with liquid nitrogen. Protein extracts were then prepared. To quantify the phospho-MET/total MET levels, a MSD 96-well MULTI-SPOT Phospho (Tyr1349)/Total MET Assay (Meso Scale Discovery) was used according to the manufacturer's instructions

Concentrations of Cpd. B and BGJ398 in plasma and tumor homogenisate were determined simultaneously by an UPLC/MS-MS assay. Following addition of 25 μl of internal standard mixture (1 μg/ml) to analytical aliquots (25 μl) of plasma or (100 μl) tumor homogenate the proteins were precipitated by the addition of 200 μl acetonitrile. The supernatant were transferred in a fresh vial. After evaporation to dryness the samples were re-dissolved in 60 μl acetonitrile/ water (1/1 v/v). An aliquot (5 μl) of this solution was separated on a ACQUITY UPLC BEH C18 columnwith a mobile phase consisting of a mixture of 0.1% formic acid in water (solvent A) and 0.1% formic acid in acetonitrile (solvent B). Gradient programming was used with a flow rate of 600 μl/min. After equilibration with 95% solvent A, 5 μl of sample was injected. Following a latency period of 0.25 min, the sample was eluted with a linear gradient of 5-100% solvent B over a period of 0.65 minutes followed by a 0.35 minutes hold. The column was prepared for the next sample by re-equilibrating over 0.25 minutes to the starting conditions. The column eluent was directly introduced into the ion source of the triple quadrupole mass spectrometer TQD™ controlled by Masslynx™ 4.1 software. Electrospray positive ionization (ESI+) multiple reaction monitoring was used for the MS/MS detection of the analyte. The limit of quantification (LOQ) for both compounds was set to 2 ng/mL and 1 ng/g for plasma and tumor homogenisate, respectively (CV and overall bias less than 30%). Regression analysis and further calculations were performed using QuanLynx™ 4.1 and Excel™ 2007. Concentrations of unknown samples were back-calculated based on the peak area ratios of analyte/IS from a calibration curve constructed using calibration samples spiked in blank plasma or tissue obtained from animals treated with vehicle.

Results: We identified a lung cancer model that displayed exceptionally high FGFR1 expression combined with high MET and HGF expression. Mice bearing xenografts derived from this model were randomized to 4 groups that were then treated with a vehicle control, Cpd. B as single agent, BGJ398 as single agent or a combination of both drugs. Cpd. B was commenced at a dose of 10 mg/kg twice daily. BGJ398 was given orally at a dose of 40 mg/kg once daily, and the same regimen for both drugs was used in combination. Due to body weight loss in the combination group, dosing frequency of Cpd. B was arbitrarily reduced to once daily after 2 weeks. The study was continued in this setup until day 18.

Compared to vehicle control, Cpd. B alone had only a very modest, statistically not significant anti-tumor effect (FIG. 8A). In contrast, treatment with BGJ398 as single agent led to strong tumor growth inhibition resulting in stable disease (stasis or slight regression) over a course of 18 days. The combination of both RTK inhibitors was able to substantially regress tumors. Statistical analysis using the method of Clarke indicated synergy.

TABLE
Anti-tumor effects and assessment of synergy

| | | A | B | AB | | | | | | |
|---|---|---|---|---|---|---|---|---|---|---|
| | C | (INC280) | (BGJ398) | (combo) | A/C | B/C | A/C × B/C | A × B/C | Difference | Result |
| Δ tumor | 309.6 | 333.6 | −16.6 | −67.8 | 1.078 | −0.054 | −0.058 | −0.219 | −0.16 | synergy |
| volume | | | | | | | | | | |

The significance of combination data was assessed using the method presented by Clark (2), which can estimate interactions from limited data. For compound A, B or the combination AB (with control group C), the end values are collected. Antagonism is predicted when the calculation AB/C>A/C×B/C, additive AB/C=A/C×B/C, synergistic interactions are predicted to occur when A×B/C>A/C×B/C.

Similar in vitro results were obtained in the cell line MG-63.

1. A pharmaceutical combination comprising (i) a MET inhibitor and (ii) an FGFR inhibitor, or a pharmaceutically acceptable salt thereof, respectively, or a prodrug thereof, respectively, and at least one pharmaceutically acceptable carrier.

2. The pharmaceutical combination according to claim 1 for simultaneous, separate or sequential use of the components (i) and (ii).

3. The pharmaceutical combination according to claim 1 in the form of a fixed combination.

4. The pharmaceutical combination according to claim 1 in the form or a kit of parts for the combined administration where the FGFR tyrosine kinase inhibitor and the MET tyrosine kinase inhibitor may be administered independently at the same time or separately within time intervals, especially where these time Intervals allow that the combination partes are jointly active.

5. The pharmaceutical combination according to claim 1, wherein the MET tyrosine kinase inhibitor is selected from the group consisting of (E)-2-(1-(3-((7-fluoroquinolin-6-yl)methyl)imidazo[1,2-b]pyridazin-6-yl)ethylidene)hydrazinecarboxamide and 2-fluoro-N-methyl-4-[7-quinolin-6-yl-methyl)-imidazo[1,2-b]triazin-2-yl]benzamide, or a pharmaceutically acceptable salt or prodrug thereof, respectively, and the FGR-R tyrosine kinase inhibitor is 3-(2,6-dichloro-3,5-dimethoxy-phenyl)-1-{6-[4-(4-ethylpiperazin-1-yl)-phenylamino]-pyridin-4-yl}-1-methyl-urea, or a pharmaceutically acceptable salt or prodrug thereof.

6. The pharmaceutical combination according to claim 1 comprising a further co-agent, or a pharmaceutically acceptable salt or a prdodrug thereof.

7. The pharmaceutical combination according to claim 1 comprising a quantity which is jointly therapeutically effective against an FGFR tyrosine kinase activity and/or MET tyrosine kinase activity mediated disease for use in the treatment of cancer.

8. The pharmaceutical combination according to claim 1 in the form of a combination product.

9. A MET Inhibitor and an FGFR inhibitor, or a pharmaceutically acceptable salt thereof, for combined use in a method of treating an FGFR tyrosine kinase activity and/or MET tyrosine kinase activity mediated disease, especially a cancer.

10. The MET inhibitor and the FGFR Inhibitor for use according to claim 9, where the MET tyrosine kinase, inhibitor is selected from the group consisting of (E)-2-(1-(3-((7-fluoroquinolin-6-yl)methyl)imidazo[1,2-b]pyridazin-6-yl)ethylidene)hydrazinecarboxamide and 2-fluro-N-methyl-4-[(7-quinolin-6-yl-methyl)-imidazo[1,2-b]triazin-2-yl]benzamide, or a pharmaceutically acceptable salt or prodrug thereof, respectively, and the FGR-R inhibitor is 3-(2,6-dichloro-3,5-dimethoxy-phenyl)-1-{6-[4-(4-ethylpiperazin-1-yl)-phenylamino]-pyrimidin-4-yl}-1-methyl-urea, or a pharmaceutically acceptable salt or prodrug thereof.

11. The use of a combination or combination product according to claim 1 for treating an FGFR tyrosine kinase activity and/or MET tyrosine kinase activity mediated disease, especially a cancer.

12. A combination of (i) an FGFR tyrosine Kinase inhibitor and (ii) a MET tyrosine kinase inhibitor or, respectively, a pharmaceutically acceptable salt thereof, for the manufacture of a medicament or a pharmaceutical product especially a combination or combination product according to claim 1, for treating an FGFR tyrosine kinase activity and/or MET tyrosine kinase activity mediated disease, especially a cancer.

13. A method of treating an FGFR tyrosine kinase activity and/or MET tyrosine kinase activity mediated disease, especially a cancer, comprising administering to a patients in need a combination of (i) an FGFR tyrosine kinase inhibitor and (ii) a MET tyrosine kinase inhibitor or, respectively, a pharmaceutical acceptable salt thereof.

14. The method according to claim 13, wherein said MET inhibitor is selected from the group consisting of (E)-2-(1-(3-((7-fluoroquinolin-6-yl)methyl)imidazo[1,2-b]pyridazin-6-yl)ethylidene)hydrazinecarboxamide and 2-fluoro-N-methyl-4-[(7-quinolin-6-yl-methyl)-imidazo[1,2-b]triazin-2-yl]benzamide, or a pharmaceutically acceptable salt or prodrug thereof, respectively. And said FGFR inhibitor is 3-(2,6-dichloro-3,5-dimathoxy-phenyl)-1-(6-[4-(4-ethyl-piperazin-1-yl)-phenylamino]-pyrimidin-4-yl)-1-methyl-urea, or a pharmaceutically acceptable salt or prodrug thereof.

15. A method for tie treatment of an FGFR tyrosine kinase activity and/or MET tyrosine kinase activity mediated disease, especially a cancer, said method comprising administering an effective amount of a combination or a combination product according to claim 1 comprising (i) an FGFR tyrosine kinase inhibitor and (ii) a MET tyrosine kinase inhibitor to a subject in need thereof, such as a warm-blocked animal, in particular a human.

16. A pharmaceutical product or s commercial package comprising a combination according to claim 1, in particular together with instructions for simultaneous, separate or sequential use thereat in the treatment of an FGFR tyrosine kinase activity and/or MET tyrosine kinase activity mediated disease, especially a cancer, in particular for use in the treatment of an FGFR tyrosine kinase activity and/or MET tyrosine kinase activity mediated disease, especially a cancer.

17. The use of (i) an FGFR tyrosine kinase inhibitor and (ii) a MET tyrosine kinase inhibitor or, respectively, a pharmaceutically acceptable salt thereof, for the preparation of a combination, especially according to any one of claims 1 to 8, for the treatment of an FGFR tyrosine kinase activity and/or MET tyrosine kinase activity mediated disease, especially a cancer.
